SEQUENCE LISTING (22 sequences)

SEQ ID NO: 1 - protein, 196 aa, Coptis japonica
MRMEVVLVVFLMFIGTINCERLIFNGRPLLHRVTKEETVMLYHELEVAASADEVWSVEGSPELGLHLPDLLPAGIFAKFEITGDGGEGSILDMTFPPGQFPHHYREKFVFFDHKNRYKLVEQIDGDFFDLGVTYYMDTIRVVATGPDSCVIKSTTEYHVKPEFAKIVKPLIDTVPLAIMSEAIAKVVLENKHKSSE

SEQ ID NO: 2 - DNA, 591 bp, Coptis japonica
  source 1..591: mol_type = other DNA
atgaggatggaagttgttctagttgttttcttgatgttcataggtacgataaattgtgaaagattgatattcaatggacgaccgctactccatcgcgtaacaaaagaggagactgtaatgctttatcatgagctggaagtagctgcttcagccgatgaagtgtggagtgtcgaaggttcgcctgagttgggcttgcatttgcctgacttgctccctgctggtatatttgcaaagtttgaaattactggtgatggaggtgaaggttcgatcctggacatgacattccccccaggtcagtttccacatcattacagggagaagttcgtgttcttcgatcacaagaatcgttacaagttagtagaacagatcgatggtgattttttcgatctaggtgttacatactatatggatacaatccgagttgttgcgacaggccctgattcatgtgtcatcaagtctactactgaataccatgtgaaacctgagtttgccaaaatcgtcaaaccacttattgacactgttccactagctatcatgtctgaagcgattgcaaaggttgttctagagaacaaacacaagagttcagagtaa

SEQ ID NO: 3 - DNA, 591 bp, Coptis japonica
  source 1..591: mol_type = other DNA
atgagaatggaagtcgtcttggtcgttttcttgatgttcattggtactatcaactgcgaaagattgatcttcaatggtagacctttgttgcacagagttaccaaagaagaaaccgttatgttgtaccacgaattggaagttgctgcttctgctgatgaagtttggtctgttgaaggttctccagaattgggtttacatttgccagatttgttgccagctggtatttttgccaagttcgaaattactggtgatggtggtgaaggttccattttggatatgacttttccaccaggtcaattcccacatcattacagagaaaagttcgtctttttcgaccacaagaacagatacaagttggtcgaacaaatcgatggtgatttcttcgatttgggtgttacttactacatggacaccattagagttgttgctactggtccagattcttgcgttattaagtctactactgaataccacgtcaagccagaatttgctaaaatcgttaagccattgatcgataccgttccattggctattatgtctgaagctattgccaaggttgtcttggaaaacaaacacaagtcatctgaatga

SEQ ID NO: 4 - protein, 6 aa, Coptis japonica
PAGIFA

SEQ ID NO: 5 - protein, 11 aa, Coptis japonica
TVPLAIMSEAI

SEQ ID NO: 6 - protein, 499 aa, Beta vulgaris
MDHATLAMILAILFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTLEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRYGS

SEQ ID NO: 7 - DNA, 1500 bp, Beta vulgaris
  source 1..1500: mol_type = other DNA
atggatcatgctaccttggctatgattttggccatcttgttcattagcttccacttcatcaagctgttgttctctcaacaaactaccaagttgttgccaccaggtccaaaaccattgccaattattggtaacatcttggaggttggtaagaagccacatagatcttttgctaatttggccaagattcacggtccattgatttctttgagattgggttctgttacgaccatcgttgtttcttcagctgatgttgctaaagagatgttcttgaagaaggatcacccattgtccaacagaactattccaaattctgttaccgctggtgatcatcataagttgactatgtcttggttgccagtttctccaaagtggcgtaatttcagaaagattaccgctgttcatttgttgtccccacaaagattggatgcttgtcaaacttttagacacgctaaggttcaacagttgtacgaatacgttcaagaatgtgctcaaaaaggtcaagccgttgatattggtaaagctgcttttactacctccttgaacttgctgtctaagttgttcttctctgttgaattggctcatcacaagtctcatacctctcaagaattcaaagagctgatctggaacatcatggaagatatcggtaagccaaattacgctgattacttcccaattttgggttgcgttgatccatctggtattagaagaagattggcttgctctttcgataagttgattgctgttttccaaggtatcatctgtgaaagattagccccagattcttctactactacaactactaccactgatgatgttttggatgtgttgttgcagttgttcaagcaaaacgaattgaccatgggtgaaatcaaccacttgttggttgatattttcgatgctggtactgataccacttcctctactttggaatgggttatgaccgaattgatcagaaacccagaaatgatggaaaaggcccaagaagagattaagcaagttttgggtaaagacaagcagatccaagaatccgatattatcaacttgccatacttgcaggccatcatcaaagaaacattgagattgcatccaccaaccgtttttttgttgccaagaaaagctgataccgatgttgagttgtatggttacatcgttccaaaggatgcccaaatcttggttaatttgtgggctattggtagagatccaaatgcttggcaaaacgccgatattttctcaccagaaaggttcattggttgcgaaattgatgttaagggtagagactttggtttgttgccttttggtgctggtagaaggatttgtccaggtatgaatttggctatcagaatgttgactttgatgctggctactctgttgcaatttttcaactggaaattggagggtgacatctcaccaaaagatttggatatggacgaaaagttcggtatcgccttgcaaaaaactaagccattgaagttgatccccattcctagatacggttcttga

SEQ ID NO: 8 - protein, 470 aa, Pseudomonas putida
MTPEQFRQYGHQLIDLIADYRQTVGERPVMAQVEPGYLKAALPATAPQQGEPFAAILDDVNNLVMPGLSHWQHPDFYGYFPSNGTLSSVLGDFLSTGLGVLGLSWQSSPALSELEETTLDWLRQLLGLSGQWSGVIQDTASTSTLVALISARERATDYALVRGGLQAEPKPLIVYVSAHAHSSVDKAALLAGFGRDNIRLIPTDERYALRPEALQAAIEQDLAAGNQPCAVVATTGTTTTTALDPLRPVGEIAQANGLWLHVDSAMAGSAMILPECRWMWDGIELADSVVVNAHKWLGVAFDCSIYYVRDPQHLIRVMSTNPSYLQSAVDGEVKNLRDWGIPLGRRFRALKLWFMLRSEGVDALQARLRRDLDNAQWLAGQVEAAAEWEVLAPVQLQTLCIRHRPAGLEGEALDAHTKGWAERLNASGAAYVTPATLDGRWMVRVSIGALPTERGDVQRLWARLQDVIKG

SEQ ID NO: 9 - DNA, 1413 bp, Pseudomonas putida
  source 1..1413: mol_type = other DNA
atgaccccagaacaattcagacaatacggtcaccaattgattgatttgatcgccgattacagacaaaccgttggtgaaagaccagttatggctcaagttgaaccaggttatttgaaagctgctttgccagctactgctccacaacaaggtgaaccatttgctgctattttggatgatgttaacaacttggttatgccaggtttgtctcattggcaacatccagatttttacggttactttccatccaacggtactttgtcatctgttttgggtgatttcttgtctactggtttgggtgttttaggtttgtcatggcaatcttctccagctttgtctgaattggaagaaactactttggattggttgagacagttgttgggtttatctggtcaatggtctggtgttattcaagatactgcttctacttctaccttggttgctttgatttctgctagagaaagagctactgattacgctttggttagaggtggtttacaagctgaacctaaaccattgatcgtttacgtttctgctcatgcccattcttcagttgataaggctgctttgttggctggttttggtagagataacattagattgattccaaccgacgaaagatacgctttaagaccagaagccttgcaagctgctattgaacaagatttggctgctggtaatcaaccatgtgctgttgttgctactactggtactactactacaactgctttggatccattaagacctgtaggtgaaattgctcaagctaatggtttgtggttgcatgttgattcagctatggctggttctgctatgattttgccagaatgtagatggatgtgggatggtattgaattggctgattctgttgttgttaacgcccataagtggttgggtgttgcttttgattgctctatctactacgttagagatccacaacacttgatcagagtgatgtctactaatccatcctacttgcaatcagctgttgatggtgaagttaagaacttgagagattggggtattccattgggtagaagatttagagctttgaagttgtggtttatgttgagatccgaaggtgttgatgcattgcaagctagattgagaagagatttggataatgctcaatggttggctggacaagttgaagctgctgctgaatgggaagttttggctccagttcaattgcaaaccttgtgcattagacatagaccagcaggtttggaaggtgaagccttggatgctcatacaaaaggttgggctgaaagattgaatgcttctggtgctgcttatgttactccagctactttagatggaagatggatggttagagtttccattggtgctttaccaactgaaagaggtgacgttcaaagattgtgggctagattgcaagatgttatcaagggttga

SEQ ID NO: 10 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTLHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 11 - DNA, 1113 bp, Saccharomyces cerevisiae
  source 1..1113: mol_type = other DNA
atgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatctagaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaattgtttgtcaacttgacaaatatcggtttgccaattggttctgaaatgcttgataccatttctcctcaatacttggctgatttggtctccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactttgcatggtgttgctgctatcaccactactaagggtaacgaacactgcttcgttattctaagaggtggtaaaaagggtaccaactacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaatag

SEQ ID NO: 12 - protein, 196 aa, Coptis japonica; identical to SEQ ID NO: 1

SEQ ID NO: 13 - DNA, 591 bp, Coptis japonica; identical to SEQ ID NO: 3
  source 1..591: mol_type = other DNA

SEQ ID NO: 14 - protein, 178 aa, Coptis japonica
MERLIFNGRPLLHRVTKEETVMLYHELEVAASADEVWSVEGSPELGLHLPDLLPAGIFAKFEITGDGGEGSILDMTFPPGQFPHHYREKFVFFDHKNRYKLVEQIDGDFFDLGVTYYMDTIRVVATGPDSCVIKSTTEYHVKPEFAKIVKPLIDTVPLAIMSEAIAKVVLENKHKSSE

SEQ ID NO: 15 - DNA, 537 bp, Coptis japonica
  source 1..537: mol_type = other DNA
atggaaagattgatcttcaatggtagacctttgttgcacagagttaccaaagaagaaaccgttatgttgtaccacgaattggaagttgctgcttctgctgatgaagtttggtctgttgaaggttctccagaattgggtttacatttgccagatttgttgccagctggtatttttgccaagttcgaaattactggtgatggtggtgaaggttccattttggatatgacttttccaccaggtcaattcccacatcattacagagaaaagttcgtctttttcgaccacaagaacagatacaagttggtcgaacaaatcgatggtgatttcttcgatttgggtgttacttactacatggacaccattagagttgttgctactggtccagattcttgcgttattaagtctactactgaataccacgtcaagccagaatttgctaaaatcgttaagccattgatcgataccgttccattggctattatgtctgaagctattgccaaggttgtcttggaaaacaaacacaagtcatctgaatga

SEQ ID NO: 16 - protein, 200 aa, Coptis japonica
MRMEVVLVVFLMFIGTINCERLIFNGRPLLHRVTKEETVMLYHELEVAASADEVWSVEGSPELGLHLPDLLPAGIFAKFEITGDGGEGSILDMTFPPGQFPHHYREKFVFFDHKNRYKLVEQIDGDFFDLGVTYYMDTIRVVATGPDSCVIKSTTEYHVKPEFAKIVKPLIDTVPLAIMSEAIAKVVLENKHKSSEHDEL

SEQ ID NO: 17 - DNA, 603 bp, Coptis japonica
  source 1..603: mol_type = other DNA
atgagaatggaagtcgtcttggtcgttttcttgatgttcattggtactatcaactgcgaaagattgatcttcaatggtagacctttgttgcacagagttaccaaagaagaaaccgttatgttgtaccacgaattggaagttgctgcttctgctgatgaagtttggtctgttgaaggttctccagaattgggtttacatttgccagatttgttgccagctggtatttttgccaagttcgaaattactggtgatggtggtgaaggttccattttggatatgacttttccaccaggtcaattcccacatcattacagagaaaagttcgtctttttcgaccacaagaacagatacaagttggtcgaacaaatcgatggtgatttcttcgatttgggtgttacttactacatggacaccattagagttgttgctactggtccagattcttgcgttattaagtctactactgaataccacgtcaagccagaatttgctaaaatcgttaagccattgatcgataccgttccattggctattatgtctgaagctattgccaaggttgtcttggaaaacaaacacaagtcatctgaacatgatgaattgtga

SEQ ID NO: 18 - protein, 196 aa, Coptis japonica
MRMEVVLVVFLMFIGTINCERLIFNGRPLLHRVTKEETVMLYHELEVAASADEVWSVEGSPELGLHLPDLLPAGIFAKFEITGDGGEGSILDMTFPPGQFPHHYREKFVFFDHKNRYKLVEQIDGDFFDLGVTYYMDTIRVVATGPDSCVIKSTTEYHVKPEFAKIVKPLIDTVPLADMSEAIAKVVLENKHKSSE

SEQ ID NO: 19 - DNA, 591 bp, Coptis japonica
  source 1..591: mol_type = other DNA
atgagaatggaagtcgtcttggtcgttttcttgatgttcattggtactatcaactgcgaaagattgatcttcaatggtagacctttgttgcacagagttaccaaagaagaaaccgttatgttgtaccacgaattggaagttgctgcttctgctgatgaagtttggtctgttgaaggttctccagaattgggtttacatttgccagatttgttgccagctggtatttttgccaagttcgaaattactggtgatggtggtgaaggttccattttggatatgacttttccaccaggtcaattcccacatcattacagagaaaagttcgtctttttcgaccacaagaacagatacaagttggtcgaacaaatcgatggtgatttcttcgatttgggtgttacttactacatggacaccattagagttgttgctactggtccagattcttgcgttattaagtctactactgaataccacgtcaagccagaatttgctaaaatcgttaagccattgatcgataccgttccattggctgatatgtctgaagctattgccaaggttgtcttggaaaacaaacacaagtcatctgaatga

SEQ ID NO: 20 - protein, 200 aa, Coptis japonica
MRMEVVLVVFLMFIGTINCERLIFNGRPLLHRVTKEETVMLYHELEVAASADEVWSVEGSPELGLHLPDLLPAGIFAKFEITGDGGEGSILDMTFPPGQFPHHYREKFVFFDHKNRYKLVEQIDGDFFDLGVTYYMDTIRVVATGPDSCVIKSTTEYHVKPEFAKIVKPLIDTVPLADMSEAIAKVVLENKHKSSEHDEL

SEQ ID NO: 21 - DNA, 603 bp, Coptis japonica
  source 1..603: mol_type = other DNA
atgagaatggaagtcgtcttggtcgttttcttgatgttcattggtactatcaactgcgaaagattgatcttcaatggtagacctttgttgcacagagttaccaaagaagaaaccgttatgttgtaccacgaattggaagttgctgcttctgctgatgaagtttggtctgttgaaggttctccagaattgggtttacatttgccagatttgttgccagctggtatttttgccaagttcgaaattactggtgatggtggtgaaggttccattttggatatgacttttccaccaggtcaattcccacatcattacagagaaaagttcgtctttttcgaccacaagaacagatacaagttggtcgaacaaatcgatggtgatttcttcgatttgggtgttacttactacatggacaccattagagttgttgctactggtccagattcttgcgttattaagtctactactgaataccacgtcaagccagaatttgctaaaatcgttaagccattgatcgataccgttccattggctgatatgtctgaagctattgccaaggttgtcttggaaaacaaacacaagtcatctgaacatgatgaattgtga

SEQ ID NO: 22 - protein, 4 aa, synthetic construct
  REGION 1..4: note = Synthetic peptide
HDEL

(19) United States
(12) Patent Application Publication
(10) Pub. No.: US 2025/0179543 A1
(43) Pub. Date: Jun. 5, 2025

(54) NORCOCLAURINE SYNTHASES WITH INCREASED ACITVITY

(71) Applicant: RIVER STONE BIOTECH, INC., Wilmington, DE (US)

(72) Inventors: Markus SCHWAB, Reinach (CH); Franziska GRASSINGER, Reinach (CH); Laura OCCHIPINTI, Reinach (CH); Philipp Friedrich BERNINGER, Reinach (CH); Jon Richard HEAL, Reinach (CH); Joseph Michael SHERIDAN, Reinach (CH); Anaelle HATSCH, Reinach (CH); Jens HOUGHTON-LARSEN, Copenhagen (DK)

(21) Appl. No.: 18/974,736

(22) Filed: Dec. 9, 2024

Related U.S. Application Data
(63) Continuation of application No. 16/623,053, filed on Dec. 16, 2019, now Pat. No. 12,203,118.

(30) Foreign Application Priority Data
Jun. 16, 2017 (DK) PA201770474
Jun. 30, 2017 (DK) PA201770533

(51) Int. Cl. C12P 17/16; C12N 9/88

(52) U.S. Cl. CPC C12P 17/165; C12N 9/88; C12Y 402/01078

(57) ABSTRACT

The invention relates to norcoclaurine synthases and substrate binding sites having one or more site-specific mutation which increase the activity, when compared to the wild type synthase, of the condensation of 4-HPAA and dopamine to (S)-norcoclaurine and/or 3,4-DhPAA and dopamine to (S)-norlaudanosoline. The inventors both identified specific mutations corresponding to at position 73, 75, 77, 82, 99, 114, 141, 142, 147, 152, 174 and/or 178 in the count according to SEQ ID No: 1, and sites corresponding to the binding domains defined in SEQ IN NO: 4 and 5, where the mutated increase of the activity may be positioned within these norcoclaurine synthases. These domains are conserved regions.

### CROSS REFERENCE TO RELATED APPLICATIONS

This application is a continuation of U.S. application Ser. No. 16/623,053, filed Dec. 16, 2019, which is U.S. national phase application under 35 U.S.C. § 371 of International Application No. PCT/EP2018/066153 filed on Jun. 18, 2018, which claims the benefit of Danish Patent Application PA 201770474 filed on Jun. 16, 2017 and Danish Patent Application No. PA 201770533 filed on Jun. 30, 2017, the disclosure of each of which is incorporated by reference in its entirety.

### SEQUENCE LISTING STATEMENT

This application contains a sequence listing which is submitted electronically and is hereby incorporated by reference in its entirety. The sequence listing submitted herewith is contained in the XML filed created Dec. 9, 2024 entitled “19-2316-WO-US-CON_Sequence-Listing.xml” and is 30,919 bytes in size.

### FIELD OF THE INVENTION

The invention relates to norcoclaurine synthases having an increased catalysation of the condensation of 4-HPAA and dopamine to (S)-norcoclaurine and/or 3,4-DhPAA and dopamine to (S)-norlaudanosoline.

### BACKGROUND OF THE INVENTION

Norcoclaurine synthase (NCS) is an enzyme involved in committing and rate limiting steps of benzylisoquinoline (BIA) biosynthesis. The two precursor molecules 4-hydroxy-phyenylacetaldehyde (4-HPAA) and dopamine are condensed in a Pictet-Spengler reaction to form (S)-norcoclaurine. Norcoclaurine synthase can also use 3,4-dihydroxy-acetaldehyde (3,4-DHPAA) as substrate in place of 4-HPAA for the formation of (S)-norlaudanosoline.

Among the known norcoclaurine synthases the homolog from Thalictrum flavum is best studied. Both biochemical and structural data are available for this enzyme. NCS is a catalytically inefficient enzyme with KM values in the double-digit mM range. Low activity is confirmed in yeast strains producing norcoclaurine. The molar concentration of the produced norcoclaurine is about 100-fold lower than the concentration of the precursor dopamine (Narcross, et al. Trends Biotechnol. 34, 228-241 (2016) & DeLoache, W. C. et al. Nat. Chem. Biol. 11, 465-471 (2015))

A heterologous yeast cell that produces norcoclaurine is e.g. known from U.S. Pat. No. 8,975,063, which disclose wildtype norcoclaurine synthase isolated from Thalictrum flavum and wildtype norcoclaurine synthase isolated from Papaverum somniferum.

Prior art describes that norcoclaurine synthase activity so far obtained in yeast is too low for driving an efficient and commercially attractive benzylisoquinoline pathway as an alternative to e.g. plant cells. Thus, there is a need for engineering an improved norcoclaurine synthase capable of increased activity in for example, a yeast strain.

### SUMMARY OF THE INVENTION

To identify norcoclaurine synthases with increased activity in heterologous host cells such as yeast, the inventors have expressed several NCS homologs on episomal vectors gathered from different species and analyzed norcoclaurine production in the supernatant of the cultures.

Codon optimized NCS from the flower Coptis japonica was identified as the most active enzyme. Further enzyme engineering approaches to improve NCS functionality where made, leading to identification of mutation sites across the protein, which increase activity and/or substrate selectivity.

The inventors identified substrate-binding domains in which mutations enhanced NCS activity.

Thus, one aspect of the invention relates to norcoclaurine synthases comprising a substrate binding amino acid sequence, which is at least 50% similar to the substrate binding amino acid sequence SEQ ID NO: 4 and/or SEQ ID NO: 5, and wherein the substrate binding amino acid sequence(s) comprise one or more mutations increasing the norcoclaurine synthase activity compared to wild type.

In one aspect of the invention relates to norcoclaurine synthases comprising an amino acid sequence which is at least 40% identical to the amino acid sequence given in SEQ ID No: 1, and which has one or more site-specific mutation at position 73, 75, 77, 82, 99, 114, 141, 142, 147, 152, 174 and/or 178 in the count according to SEQ ID No: 1.

In another aspect, the present invention also relates to mutated norcoclaurine synthase amino acid sequences originating from Coptis japonica having an increased catalysation, when compared to the wild type synthase, of the condensation of 4-HPAA and dopamine to (S)-norcoclaurine and/or 3,4-DHPAA and dopamine to (S)-norlaudanosoline. The mutation may for example be present in the substrate-binding amino acid sequences.

### DETAILED DESCRIPTION OF THE INVENTION

For driving an efficient and commercially viable benzylisoquinoline pathway in heterologous host cells, currently known wild type norcoclaurine synthases like, for example those sourced from Coptis japonica or Thalictrum flavum do not provide a high enough activity via the Pictet-Spengler reaction to form (S)-norcoclaurine in sufficient quantities.

Many benzylisoquinoline alkaloids are of great medical interest because of their pharmacological activity, such as, for example, the antibiotic sanguinarine, the muscle relaxants papaverine and tubocurarine and the analgesics codeine and morphine.

Galanie S. et al., Science 2015 disclosed a complete biosynthesis of opioids through enzyme discovery, enzyme engineering, and pathway and strain optimization to realize full opiate biosynthesis in yeast, thereby enabling obtaining all of the following end-products: Thebaine, Oripavine, Neopinone, Codeinone, Hydrocodone, Morphine, Oxycodone and Codeine. Furthermore, DeLoache, W. C. et al., Nature Chemical

Biology 2015 disclosed the pathway to fully reconstitute the seven-enzyme pathway from L-tyrosine to (S)-reticuline, thereby enabling obtaining all of the following end-products: Noscapine, Berberine, Sanguinarine, Tubocurarine and Papaverine. Thus, the present invention relates to improved production of any of the above-mentioned opioids in yeast via mutated norcoclaurine synthase as described herein.

The invention describes the engineering of norcoclaurine synthase having site-specific mutations for increased activity in heterologous host cells. These site-specific mutations may additionally be transferred between species due to their unique positions in the norcoclaurine synthase, because the mutation sites are sited in putative substrate binding domains whose relative positions will be conserved between different NCS enzymes, such as but not limited to conserved domains amongst species. The conserved domains can for example be the helix structures and/or loop binding sites identified by the inventors.

In one aspect the invention relates to a norcoclaurine synthase comprising a substrate-binding amino acid sequence, which is at least 50% similar or 20% identical to the substrate binding amino acid sequence SEQ ID NO: 4 and/or SEQ ID NO: 5, and wherein the substrate binding amino acid sequence(s) comprise one or more mutations increasing the norcoclaurine synthase activity compared to wild type. In some embodiments, the substrate binding amino acid sequence identified by the inventors and disclosed herein is an amino acid sequence which is at least 50% similar or 20% identical to the amino acid sequence SEQ ID NO: 4.

In some embodiments, another substrate binding amino acid sequence identified is an amino acid sequence, which is at least 50% similar or 20% identical to the amino acid sequence SEQ ID NO: 5. Both of these substrate-binding amino acid sequences are engineered to comprise one or more mutations increasing the norcoclaurine synthase activity compared to wild type.

In the present context, the term substrate binding amino acid sequence, substrate binding site or catalytic site may be used interchangeably.

### Norcoclaurine Synthases (NCS)

Norcoclaurine synthases (NCS) are enzymes capable of catalysing the formation of (S)-norcoclaurine and (S)-norlaudonosoline via condensation of 4-HPAA and dopamine (for norcoclaurine) or 3,4-DHPAA and dopamine (for norlaudonosoline, see FIG. 1).

This enzyme belongs to the family of lyases, specifically the hydrolyases, which cleave carbon-oxygen bonds. The systematic name of this enzyme class is 4-hydroxyphenylacetaldehyde hydro-lyase. Other names in common use include (S)-norlaudanosoline synthase, and 4-hydroxyphenylacetaldehyde hydro-lyase. This enzyme participates in benzylisoquinoline alkaloid (BIA) biosynthesis.

The NCS of the present invention have amino acid sequences, which are at least 40% identical to the amino acid sequence given in SEQ ID No: 1. However, the NCS of the invention do not relate to the wild type NCS in SEQ ID NO: 1 per se.

In some embodiments, the mutation(s) may comprise one or more site-specific mutations corresponding to position 2, 4, or 6 in the count according to SEQ ID No: 4 and/or position 2, or 6 in the count according to SEQ ID No: 5. In some embodiments, the mutation(s) may further comprise one or more site-specific mutation corresponding to position 73, 75, 77, 174 and/or 178 in the count according to SEQ ID No: 3.

The NCS disclosed herein have an increased catalysation of the condensation of 4-HPAA and dopamine to (S)-norcoclaurine and/or 3,4-DhPAA and dopamine to (S)-norlaudanosoline, when compared to the wild type NCS in SEQ ID NO: 1 per se.

NCS molecules of the invention have amino acid sequences, which are at least 40% identical to the amino acid sequence given in SEQ ID NO: 1. NCS of the invention may comprise substrate binding domains as described. However, NCS disclosed herein do not relate to the wild type NCS in SEQ ID NO: 1 per se because NCS disclosed herein have an increased catalysation of the condensation of 4-HPAA and dopamine to (S)-norcoclaurine and/or 3,4-DhPAA and dopamine to (S)-norlaudanosoline, when compared to the wild type NCS in SEQ ID NO: 1.

Accordingly, NCS variants disclosed herein are more suitable for commercial benzylisoquinoline alkaloid biosynthesis given their improved activity.

As used herein, the term “increased catalysation” means increasing the speed of the condensation compared to the wild type NCS in SEQ ID NO: 1 per se. Terms like “catalytic activity”, “turnover”, “activity” could be used interchangeably.

### Mutation Positions

The inventors disclose specific mutations in NCS derived from Coptis japonica that results in increased activity of the condensation process. Amino acid exchanges in positions corresponding to positions 73, 75, 77, 82, 99, 114, 141, 142, 147, 152, 174 and/or 178 of SEQ ID No: 1 provided the best increase in formation of S-Norcoclaurine (g/ml), when compared to the wild type—see Table 8.

Thus, in one or more exemplary embodiments, the invention relates to a norcoclaurine synthase comprising an amino acid sequence which is at least 40% identical to the amino acid sequence given in SEQ ID No: 1, and which has one or more site-specific mutations corresponding to position 73, 75, 77, 82, 99, 114, 141, 142, 147, 152, 174 and/or 178 in the count according to SEQ ID No: 1.

The site-specific mutation corresponding to position 73 in the count according to SEQ ID No: 1 may preferably be Proline (P). Thus, in one or more exemplary embodiments, the invention relates to a norcoclaurine synthase, which has the amino acid Proline (P) corresponding to position 2 in the count according to SEQ ID No: 4. In one or more exemplary embodiments, the site-specific mutation is A73P in the count according to SEQ ID No: 1. In one or more exemplary embodiments, the site-specific mutation is A2P in the count according to SEQ ID No: 4.

In one or more exemplary embodiments the site-specific mutation corresponding to position 75 in the count according to SEQ ID No: 1 is Leucine (L) or Lysine (K). Thus, in one or more exemplary embodiments, the invention relates to a norcoclaurine synthase, which has the amino acid Leucine (L) or Lysine (K) corresponding to position 4 in the count according to SEQ ID No: 4. In one or more exemplary embodiments, the site-specific mutation is I75K or I75L in the count according to SEQ ID No: 1. In one or more exemplary embodiments, the site-specific mutation is I4K or I4L in the count according to SEQ ID No: 4.

The site-specific mutation corresponding to position 77 in the count according to SEQ ID No: 1 may preferably be Serine (S), Threonine (T) or Glutamic Acid (E). In one or more exemplary embodiments, the site-specific mutation is A77S, A77T or A77E in the count according to SEQ ID No: 1.

In one or more exemplary embodiments, the site-specific mutation is A6S, A6T or AGE in the count according to SEQ ID No: 4.

The site-specific mutation corresponding to position 82 in the count according to SEQ ID No: 1 may preferably be Valine (V). In one or more exemplary embodiments, the site-specific mutation is T82V in the count according to SEQ ID No: 1.

The site-specific mutation corresponding to position 99 in the count according to SEQ ID No: 1 may preferably be Lysine (K) or Arginine (R). In one or more exemplary embodiments, the site-specific mutation is Q99K or Q99R in the count according to SEQ ID No: 1.

The site-specific mutation corresponding to position 141 in the count according to SEQ ID No: 1 may preferably be Isoleucine (I). In one or more exemplary embodiments, the site-specific mutation is V1411 the count according to SEQ ID No: 1.

The site-specific mutation corresponding to position 142 in the count according to SEQ ID No: 1 may preferably be Isoleucine (I). In one or more exemplary embodiments, the site-specific mutation is V1421 the count according to SEQ ID No: 1.

The site-specific mutation corresponding to position 147 in the count according to SEQ ID No: 1 may preferably be Asparagine (N). In one or more exemplary embodiments, the site-specific mutation is D147N the count according to SEQ ID No: 1.

The site-specific mutation corresponding to position 152 in the count according to SEQ ID No: 1 may preferably be Arginine (R). In one or more exemplary embodiments, the site-specific mutation is K152R the count according to SEQ ID No: 1.

The site-specific mutation corresponding to position 174 in the count according to SEQ ID No: 1 may preferably be Glycine (G) or Glutamic Acid (E). In one or more exemplary embodiments, the site-specific mutation is V174G or V174E in the count according to SEQ ID No: 1. Thus, in one or more exemplary embodiments, the invention relates to a norcoclaurine synthase, which has the amino acid Glycine (G) or Glutamic Acid (E) corresponding to position 2 in the count according to SEQ ID No: 5.

In one or more exemplary embodiments, the site-specific mutation is V2G or V2E in the count according to SEQ ID No: 5.

The site-specific mutation corresponding to position 178 in the count according to SEQ ID No: 1 may preferably be Alanine (A), Serine (S) or Aspartic Acid (D). In one embodiment, the site-specific mutation is I178A, I178S or I178D in the count according to SEQ ID No: 1. Thus, in one embodiment, the invention relates to a norcoclaurine synthase, which has the amino acid Alanine (A), Serine (S) or Aspartic Acid (D) corresponding to position 6 in the count according to SEQ ID No: 5. In one embodiment, the site-specific mutation is I6A, I6S or I6D in the count according to SEQ ID No: 5.

The site-specific mutations described above may be combined in double, triple or further mutations to enhance the effect of the norcoclaurine synthase. The skilled addressee can test these combinations by the methods disclosed in the Examples, which are intended to provide non-limiting examples of mutations and combinations of mutations. All such combinations are individual embodiments of the present invention regardless of the corresponding position count according to SEQ ID NO: 1-5, and various of these embodiments are described below.

In one or more exemplary embodiments, the invention relates to a norcoclaurine synthase, which has the amino acid Proline (P) corresponding to position 73 and Glutamic Acid (E) corresponding to position 77 in the count according to SEQ ID No: 1, e.g. A73P/A77E.

In one or more exemplary embodiments, the invention relates to a norcoclaurine synthase, which has the amino acid Proline (P) corresponding to position 2 and Leucine (K) or Lysine (L) corresponding to position 4 in the count according to SEQ ID No: 4, e.g. A2P/I4K, A2P/I4L.

In one or more exemplary embodiments, the invention relates to a norcoclaurine synthase, which has the amino acid Arginine (R) corresponding to position 152 and Glutamic Acid (E) corresponding to position 174 in the count according to SEQ ID No: 1, e.g. K152R/V174E.

In one or more exemplary embodiments, the invention relates to a norcoclaurine synthase, which has the amino acid Proline (P) corresponding to position 2 and Serine (S), Threonine (T) or Glutamic Acid (E) corresponding to position 6 in the count according to SEQ ID No: 4, e.g. A2P/A6S, A2P/A6T, or A2P/A6E.

In one or more exemplary embodiments, the invention relates to a norcoclaurine synthase, which has the amino acid Glutamic Acid (E) corresponding to position 114 and Glutamic Acid (E) corresponding to position 174 in the count according to SEQ ID No: 1, e.g. K114E/V174E.

In one or more exemplary embodiments, the invention relates to a norcoclaurine synthase, which has the amino acid Leucine (K) or Lysine (L) corresponding to position 4 and Serine (S), Threonine (T) or Glutamic Acid (E) corresponding to position 6 in the count according to SEQ ID No: 4, e.g. I4K/A6S, I4K/A6T, I4K/A6E, I4L/A6S, I4L/A6T, or I4L/A6E.

In one or more exemplary embodiments, the invention relates to a norcoclaurine synthase, which has the amino acid Glutamic Acid (E) corresponding to position 114 and Glycine (G) corresponding to position 174 in the count according to SEQ ID No: 1, e.g. V174G/K114E.

In one or more exemplary embodiments, the invention relates to a norcoclaurine synthase, which has the amino acid Proline (P) corresponding to position 2, Leucine (K) or Lysine (L) corresponding to position 4 and Serine (S), Threonine (T) or Glutamic Acid (E) corresponding to position 6 in the count according to SEQ ID No: 4, A2P/I4K/A6S, A2P/I4K/A6T, A2P/I4K/A6E, A2P/I4L/A6S, A2P/I4L/A6T, or A2P/I4L/A6E.

In one or more exemplary embodiments, the invention relates to a norcoclaurine synthase, which has the amino acid Arginine (R) corresponding to position 152 and Glycine (G) corresponding to position 174 in the count according to SEQ ID No: 1, e.g. V174G/K152R.

In one or more exemplary embodiments, the norcoclaurine synthase may have site-specific mutations at both position 2 and 6 in the count according to SEQ ID No: 5.

In one or more exemplary embodiments, the invention relates to a norcoclaurine synthase, which has the amino acid Arginine (R) corresponding to position 152 and Alanine (A) or Aspartic Acid (D) corresponding to position 178 in the count according to SEQ ID No: 1, e.g. I178D/K152R or I178A/K152R.

Thus, in one or more exemplary preferred embodiments, position 2 and 6 in the count according to SEQ ID No: 5 has the amino acid Glycine (G) or Glutamic Acid (E) corresponding to position 2 and Alanine (A), Serine (S) or Aspartic Acid (D) corresponding to position 6, e.g. V2G/I6A, V2G/I6S, V2G/I6D, V2E/I6A, V2E/I6S, or V2E/I6D in the count according to SEQ ID No: 5.

Other combinations like for example A73P/V174G, A73P/V174E, A66S/V174G, A66S/V174E in the count according to SEQ ID No: 1 is also an embodiment of the invention.

In one or more exemplary presently preferred embodiments, the combinatorial mutations may be selected from the group consisting of I178D+I75L, I178D+I75K, I178D+A77S, I178D+V174Q, A73P+A77E and A77T+I178D in the count according to SEQ ID No: 1.

In one or more exemplary presently preferred embodiments, the combinatorial mutations may be selected from the group consisting of K114E/V174E, K152R/V174E, V174G/K152R, V174G/K114E, I178A/152R, I178D/K152R, I178D/174E/K152R, I178D/K114E/K152R, I178D/V174E/Q99K, I178D/V174Q/Q99K, I178D/V174E/Q99R, and I178D/V174Q/Q99R in the count according to SEQ ID No: 1.

In one or more exemplary presently preferred embodiments, the combinatorial mutations may be selected from the group consisting of I178D+I75L, I178D+I75K, I178D+A77S, I178D+A77S+K152R, I178D+I75K+K152R, I178D+V174G+K152R+A77S, A77T+I178D, I178D+V174Q, A73P+A77E and A77T+I178D in the count according to SEQ ID No: 1.

In one or more exemplary embodiments, the invention relates to a norcoclaurine synthase, wherein the site-specific mutation is selected from the group consisting of A73P, I75L, I75K, A77S, A77E, A77T, T82V, Q99K, Q99R K114E, V1411, V1421, D147N, K152R, V174E, V174G, V174Q, V174E, I178A, I178S, I178D, I178N, I178Q, and I178T in the count according to SEQ ID No: 1.

### The In Silico Predicted Flexible Loop as Substrate Binding Domain

In addition to the helical domain described below, which is predicted to interact with substrate and may be advantageously mutated to enhance such interactions, the generated homology model of C. japonica NCS also reveals a flexible loop region, which is predicted to interact with substrates.

The inventors show that this motif also contains amino acids for which mutations affect substrate binding. This loop is formed by amino acids P72-A77 in the count corresponding to SEQ ID NO: 1—equivalent to P1-A6 in the count corresponding to SEQ ID NO: 4—and is located in 3D proximity to the helix domain (SEQ ID NO: 5). Thus, the flexible loop is also predicted to contact the substrate, based on the model of C. japonica NCS disclosed by the inventors.

In one or more presently preferred embodiments, the invention relates to mutations in this flexible loop binding domain comprising site specific mutations corresponding to position 2, 4 and 6 in the count corresponding to SEQ ID 4.

Such mutations may be advantageously combined with those from helix domain, because these two regions interact in the protein model and therefore combined mutations will have concerted effects on substrate binding.

### The In Silico Predicted Helix Structure as Substrate Binding Motif

The crystal structure of T. flavum NCS (Ilari et al. 2009, Journal of biological chemistry, 284(2), pp. 897-904), and a subsequent study (Bonamore et al. 2010, Molecules, 15(4), pp. 2070-2078) suggest an enzymatic mechanism for NCS that involves binding of 4-HPAA to the enzyme prior to dopamine (the ‘HPAA-first’ mechanism). An alternative mechanism has also been proposed in which dopamine binds to the enzyme prior to 4-HPAA (the ‘dopamine-first’ mechanism) (Lichman et al. 2015, FEBS journal, 282(6), pp. 1137-1151).

The invention relates to a norcoclaurine synthase comprising mutations in aldehyde binding pocket domains of the synthase regardless of the enzymatic mechanism.

Based on the model of C. japonica NCS disclosed by the inventors, amino acid positions V174 and I178 corresponding to SEQ ID NO: 3 (V2 and I6 in SEQ ID NO: 2) are predicted to affect the catalytic activity of NCS.

For example, the predicted location in an alpha helix with an N-terminus at the wild-type amino acid position V174 and a C-terminus corresponding to position L188. For example, position 178 of NCS contains Isoleucine. PROTOSCAN was used to rank energies of other mutations at this position and aspartic acid had the lowest energy, because of enhanced interaction with the para hydroxyl group of HPAA substrate as shown in FIG. 2.

Irrespective of the HPAA-first or dopamine-first mechanisms, this model of C. japonica NCS shows that this helix corresponding to positions 174-188 of SEQ ID NO: 3 is involved in binding of the aldehyde and/or dopamine substrates of NCS. In addition, the location of the helix corresponding to positions 174-188 of SEQ ID NO: 3 suggests that it may also gate access of aldehyde and/or dopamine substrates to the active site of NCS. Therefore, other amino acid variants of the helix corresponding to positions 174-188 in C. japonica may also affect catalytic activity.

Thus, in one or more exemplary embodiments, the present invention relates to a norcoclaurine synthase comprising an amino acid sequence forming a helix structure homologue to positions 174-188 in C. japonica, and wherein site-specific mutations in said helix structure improve the activity of the enzyme.

The invention also relates to a norcoclaurine synthase having an amino acid sequence forming a helix structure, which is at least 60% identical to the amino acid sequence given in SEQ ID No: 4, such as 65%, 70%, 75%, 80%, 85%, 95% identical. Said norcoclaurine synthase can within said helix structure further comprise at least one site-specific mutations, such as one site-specific mutation, two site-specific mutations, three site-specific mutations, four site-specific mutations or more.

In one or more exemplary embodiments, the helix structure comprises site-specific mutation(s) corresponding to position 174 and/or 178 in SEQ ID 1 (2 and 6 in SEQ ID NO: 5).

The inventors demonstrate that NCS's having mutated helical substrate binding domains with only 10 amino acid are industrial beneficial, and this domain is conserved across NCS's, which provides an effective choke point across species.

### Disruption of Signal Peptide Function

Using the amino acid sequence of the C. japonica NCS as query, a signal peptide prediction was done using SignalP 4.1. (Petersen, T. N et al Nature Methods, 8 :785-786, 2011). This program predicts that the first 19 amino acid of the C. japonica NCS constitute a signal peptide with a predicted cleavage site between amino acids 19 and 20 from the N-terminus. As seen in FIG. 7 Norcoclaurine production in S. cerevisiae increases dramatically, when the first 19 amino acids from the N-terminal of the C. japonica NCS are replaced by a methionine.

In one or more exemplary embodiments, the invention relates to a norcoclaurine synthase according to the present disclosure where the signal peptide function has been disrupted. This could typically be via mutations or partly removal of the signal peptide sequence. In fact, any change to the NCS that prevent the N-terminal signal peptide from working, i.e. any change that prevents translocation of the NCS into the ER lumen. Thus, the invention relates to a mutated norcoclaurine synthase having an increased catalysation when compared to the wild type synthase of the condensation of 4-HPAA and dopamine to (S)-norcoclaurine and/or 3,4-DHPAA and dopamine to (S)-norlaudanosoline comprising an amino acid sequence, which is at least 40% identical to the amino acid sequence given in SEQ ID No: 1, further comprising rendering the signal peptide non-functional.

Thus, in one or more exemplary embodiments, the invention relates to a norcoclaurine synthase according to the present disclosure further comprising replacing one or more amino acids from the N-terminal with a methionine or by mutation or removal of one or more amino acids from the signal peptide sequence or any truncation rendering the signal peptide non-functional.

The 19 amino acid truncation, see FIG. 7, deletes the predicted signal peptide sequence and therefore prevents the enzyme from entering the secretory pathway. Instead the enzyme will go to the cytosol instead.

Thus, in one or more exemplary embodiments, the invention relates to a norcoclaurine synthase according to the present disclosure further comprising replacing at least 1 amino acids from the N-terminal with a methionine, such as at least 1 amino acids, at least 2 amino acids, at least 3 amino acids, at least 4 amino acids, at least 5 amino acids, at least 6 amino acids, at least 7 amino acids, at least 8 amino acids, at least 9 amino acids, at least 10amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, or more amino acids from the N-terminal with a methionine.

While the plant signal peptide is very likely to work in yeast, other sorting and retention signals might not. It is therefore very likely that the full-length C. japonica NCS expressed in yeast is excreted by vesicular transport from the ER lumen through the Golgi apparatus to the plasma membrane and thereby to the extracellular space.

### Addition an ER Retention Signal to the NCS Enzyme—HDEL (SEQ ID NO:22)

The addition of the amino acids HDEL (SEQ ID No: 22) to the C-terminus of a full-length C. japonica NCS has a significant positive effect of Norcoclaurine production in S. cerevisiae. The addition of the yeast ER retention signal HDEL (SEQ ID No:22) to the C-terminus of the CjNCS was designed to retain the enzyme in the ER/golgi and thereby prevent it from being excreted into the extracellular space.

The positive effect on Norcoclaurine production of this localization is unexpected since the enzymes that produce dopamine and 4-HPAA are believed to be cytosolic and it shows that a pool of 4-HPAA and dopamine exists not only in the cytosol, but also in the ER/golgi of S. cerevisiae.

In one or more exemplary embodiments, the invention relates to a Norcoclaurine synthase according to the present disclosure, wherein the Norcoclaurine synthase is held in the ER/golgi. The effect is also achievable for unmutated Norcoclaurine synthase, thus in one or more exemplary embodiments, the invention relates to a Norcoclaurine synthase having an increased catalysation when compared to the wild type synthase of the condensation of 4-HPAA and dopamine to (S)-norcoclaurine and/or 3,4-DHPAA and dopamine to (S)-norlaudanosoline comprising an amino acid sequence, which is at least 40% identical to the amino acid sequence given in SEQ ID No: 1 and further comprising an addition of the yeast ER retention signal HDEL (SEQ ID NO:22) to the C-terminus. Localisation will typically be shown by either N-or C-terminal tagging of the protein with a fluorescent protein, GFP, RFP or similar followed by fluorescence microscopy.

Altering the localization of the NCS, thus allows the utilization of hitherto inaccessible pools of 4-HPAA and dopamine. This more complete utilization can be beneficial for both obtaining higher titers, but also to prevent potentially harmful accumulation of precursors in organelles of S. cerevisiae by conversion into later non-toxic pathway products. Dopamine is known to be cytotoxic in S. cerevisiae.

In one or more exemplary embodiments, the invention relates to a norcoclaurine synthase according to the present disclosure, further comprising addition of the amino acids HDEL (SEQ ID No:22) to the C-terminus.

Coptis japonica NCS^{I178D} With HDEL (SEQ ID No:22) Addition

As seen in FIG. 8 below the change of the amino acid I178 to a D (isoleucine to aspartic acid) of the C. japonica sequence improves the activity of the enzyme and hence the production of norcoclaurine significantly.

Moreover, FIG. 8 also shows that the CjNCS I178 to D amino acid change also significantly improves the activity of the C. japonica NCS version with the HDEL (SEQ ID No: 22) addition in the C-terminus.

Again, this shows that a large and untapped pool of dopamine and 4-HPAA exists in the ER/golgi of S. cerevisiae. Since the ΔI9-CjNCS and the CjNCS-HDEL (both native and I178D) exists in different compartments of the yeast cell, co-expression of both is likely to give effects on norcoclaurine production titers.

In one or more presently preferred exemplary embodiments, the invention relates to a norcoclaurine synthase comprising the site-specific mutation to Aspartic Acid (D) corresponding to position 178 in the count according to SEQ ID No: 1, and further comprising the addition of the amino acids HDEL (SEQ ID No:22) to the C-terminus.

### Amino Acid Sequence Identity

Sequence analysis can involve BLAST, Reciprocal BLAST, or PSI-BLAST analysis of non-redundant databases using the amino acid sequence of interest as the reference sequence. Amino acid sequence is, in some instances, deduced from the nucleotide sequence. Those polypeptides in the database that have greater than 40% sequence identity are candidates for further evaluation for suitability as polypeptide useful in the synthesis of compounds described herein. Amino acid sequence similarity allows for conservative amino acid substitutions, such as substitution of one hydrophobic residue for another or substitution of one polar residue for another. When desired, manual inspection of such candidates can be carried out in order to narrow the number of candidates to be further evaluated. Manual inspection can be performed by selecting those candidates that appear to have conserved functional domains.

Conserved regions can be identified by locating a region within the primary amino acid sequence of a polypeptide described herein that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/and pfam.janelia.org/. The information included at the Pfam database is described in Sonnhammer et al., Nucl. Acids Res., 26 : 320-322 (1998); Sonnhammer et at., Proteins, 28 :405-420 (1997); and Bateman et al., Nucl. Acids Res., 27 : 260-262 (1999). Conserved regions also can be determined by aligning sequences of the same or related polypeptides from closely related species. Closely related species preferably are from the same family. In some aspects, alignment of sequences from two different species can be adequate.

The polypeptides disclosed herein may exhibit at least 40% amino acid sequence identity to any of SEQ ID NOs: 1-3, such as at least 45% amino acid sequence identity, at least 50%, at least 60%, at least 70%, at least 80%, or at least 90% amino acid sequence identity. In some embodiments, the polypeptides exhibit at least 81%, 82%, 83%, 84%, 85%, 86%, 87%, 88%, 89%, 91%, 92%, 93%, 94%, 95%, 96%, 97%, 98%, or 99% amino acid sequence identity.

### Identity

The term “% identity” is used herein about the relatedness between two amino acid sequences or between two nucleotide sequences.

The term “% identity” as used herein about amino acid sequences means the degree of identity in percent between two amino acid sequences obtained when using the Needleman-Wunsch algorithm (Needleman and Wunsch, 1970, J. Mol. Biol. 48 : 443-453) as implemented in the Needle program of the EMBOSS package (EMBOSS: The European Molecular Biology Open Software Suite, Rice et al., 2000, Trends Genet. 16 : 276-277), preferably version 5.0.0 or later.

### Similarity

The term “% similarity” as used herein about amino acid sequences means the degree of similarity in percent between two amino acid sequences obtained when using the Needleman-Wunsch algorithm (Needleman and Wunsch, 1970, supra) as implemented in the Needle program of the EMBOSS package (EMBOSS : The European Molecular Biology Open Software Suite, Rice et al., 2000, Trends Genet. 16 : 276-277), preferably version 5.0.0 or later.

The parameters used are gap open penalty of 10, gap extension penalty of 0.5, and the EBLOSUM62 (EMBOSS version of BLOSUM62) substitution matrix. The output of Needle labeled “longest identity” (obtained using the-nobrief option) is used to calculate the percent identity follows:

100*identical amino acid residues/(Length of alignment−total number of gaps in alignment)

The output of Needle labeled “longest similarity” (obtained using the -no brief option) is used to calculate the percent similarity as follows:

100*similar amino acid residues/(Length of alignment−total number of gaps in alignment)

The protein sequences disclosed herein can further be used as a “query sequence” to perform a search against sequence databases, for example to identify other family members or related sequences. Such searches can be performed using the BLAST programs. Software for performing BLAST analyses is publicly available through the National Centre for Biotechnology Information.

BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins

Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity.

It will be appreciated that polypeptides described herein can include additional amino acids that are not involved in other enzymatic activities carried out by the enzyme, and thus such a polypeptide can be longer than would otherwise be the case. For example, a polypeptide can include a purification tag (e.g., HIS tag or GST tag), a chloroplast transit peptide, a mitochondrial transit peptide, an amyloplast peptide, signal peptide, or a secretion tag added to the amino or carboxy terminus. In some embodiments, a polypeptide includes an amino acid sequence that functions as a reporter, e.g., a green fluorescent protein or yellow fluorescent protein.

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

As disclosed herein, a “regulatory region” (prokaryotic and eukaryotic) refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof.

A regulatory region typically comprises at least a core (basal) promoter. A regulatory region also can include at least one control element, such as an enhancer sequence, an upstream element, or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements.

The polypeptides of the invention exhibit at least 20% amino acid sequence identity to any of SEQ ID NOs: 1-3, such as at least 25%, at least 30%, at least 35%, at least 40%, at least 45%, at least 50%, at least 55%, at least 60%, at least 65%, at least 70%, at least 75%, at least 80%, at least 85%, or at least 90% amino acid sequence identity.

In some embodiments, the polypeptides according to any of SEQ ID NOs: 1-3 exhibit at least 81%, 82%, 83%, 84%, 85%, 86%, 87%, 88%, 89%, 91%, 92%, 93%, 94%, 95%, 96%, 97%, 98%, or 99% amino acid sequence identity.

### Functional Homologues and Genetic Variation

Functional homologs of the polypeptides described above are also suitable for use in producing the compounds mentioned herein in a recombinant host. A functional homolog is a polypeptide that has sequence similarity to a reference polypeptide, and that carries out one or more of the biochemical or physiological function(s) of the reference polypeptide.

A functional homolog and the reference polypeptide can be natural occurring polypeptides, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”).

Techniques for modifying genes encoding functional the polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide: polypeptide interactions in a desired manner.

Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of polypeptides described herein.

Typically, polypeptides that exhibit at least about 20% amino acid sequence identity are useful to identify conserved regions. Conserved regions of related polypeptides exhibit at least 25% amino acid sequence identity e.g., at least 30%, at least 40%, at least 55%, at least 50%, at least 60%, at least 70%, at least 80%, or at least 90% amino acid sequence identity. In some aspects the invention relates to a conserved region exhibits at least 92%, 94%, 96%, 98%, or 99% amino acid sequence identity. The conserved region may be considered to be the entire protein or nucleic acid sequence.

An aspect of the invention relates to a functional homologue that has at least 20% sequence identity with an amino acid or nucleic acid sequence mentioned herein, such as 40% sequence identity, such as 50% sequence identity, such as 60% sequence identity, such as 70% sequence identity, such as 75% sequence identity, such as 80% sequence identity, such as 85% sequence identity, such as 90% sequence identity, such as 75% sequence identity, such as 97% sequence identity, such as 98% sequence identity, such as 99% sequence identity.

### Method for Testing Different Homologs

A person skilled in the art would recognize that beneficial mutations described here can be advantageously applied to sequence homologs from organisms other than Coptis japonica resulting in new variants of such homologs with altered properties.

One method to identify the equivalent mutation positions in other homologs would be to align homolog sequences to Coptis japonica NCS using a readily available protein alignment tool, and then use the resulting alignment to identify the corresponding position in the homolog at which a mutation is known to be beneficial in the CjNCS sequence SEQ ID NO: 1.

A new homolog variant(s) can then be designed by swapping the wild type amino acid at that position in the homolog with beneficial mutations described herein before synthesizing the new variants and testing for activity in a suitable assay, for example the assay for NCS activity described in this Examples 5, 6 and 7.

For example, a “I178-analog” position corresponds to position I178 according to SEQ ID NO: 1 can be determined by sequence alignment of NCS sequences from organisms other than Coptis japonica as explained herein. For example the I178-analog position of NCBI-ProteinID: [AAR22502.1] is position A182, and of NCBI-ProteinID: [ACJ76785.1] the position is S170 and of NCBI-ProteinID: [SEAAX56304.1] is S181.

Corresponding analogies apply to the other sequence positions described for SEQ ID NO: 1 herein, and include A73, 175, A77, and V174 and their analogous positions. The GenBank protein sequence reference number, the amino acid residue “corresponding” to position V174 of SEQ ID NO: 1, i.e. I178-analog (“Aa”) and whose sequence position are presented in the Table 1.

For purposes of the invention, in Table 1 below the amino acid sequence SEQ ID NO: 1 was aligned pairwise with the amino acid sequences in Table 1 determined using the Needleman-Wunsch algorithm (Needleman and Wunsch, 1970, J. Mol. Biol. 48 : 443-453) as implemented in the Needle program of the EMBOSS package (EMBOSS : The European Molecular Biology Open Software Suite, Rice et al., 2000, Trends Genet. 16 : 276-277), preferably version 3.0.0, 5.0.0, 6.6.0 or later. The Needle program of the EMBOSS package creates an optimal global alignment of two sequences using the Needleman-Wunsch algorithm. The parameters used are gap open penalty of 10, gap extension penalty of 0.5, End Gap Penalty is set as false, End Gap Open Penalty of 10.0, End Gap Extension Penalty of 0.5, and the EBLOSUM62 (EMBOSS version of BLOSUM62) substitution matrix.

Functional homologs can be identified by analysis of nucleotide and polypeptide sequence alignments. For example, nucleic acid that encodes a functionally or polypeptide sequences can identify homologs of polypeptides described herein.

In some embodiments, the coding sequence for a polypeptide described herein is identified in a species other than the recombinant host, i.e., is a heterologous gene.

Thus, if the recombinant host is a microorganism, the coding sequence can be from other prokaryotic or eukaryotic microorganisms, from plants or from animals. In some cases, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant gene construct. In addition, stably transformed exogenous genes typically are integrated corresponding to positions other than the position where the native sequence is found.

One or more genes can be combined in a recombinant nucleic acid construct in “modules” useful for a discrete aspect of production of a compound described herein. Combining a plurality of genes in a module, particularly a polycistronic module, facilitates the use of the module in a variety of species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). As isolated nucleic acids, these modified sequences can exist as purified molecules and can be incorporated into a vector or a virus for use in constructing modules for recombinant nucleic acid constructs.

TABLE 1

| | GenBank Accession | | |
|---|---|---|---|
| | numbers of the | | |
| Organism | Reference sequences | Aa | Position |
| Coptis | BAF45338.2 | I | 178 |
| japonica | | | |
| Thalictrum | AAR22502.1 | A | 182 |
| flavum | | | |
| Expression vector | AKH61495.1 | A | 164 |
| pWCD2339 | | | |
| Sinopodophyllum | AIT42265.1 | A | 175 |
| hexandrum | | | |
| Argemone | ACJ76785.1 | S | 170 |
| mexicana NCS1 | | | |
| Corydalis | AEB71889.1 | S | 156 |
| saxicola | | | |
| Argemone | ACJ76787.1 | S | 170 |
| mexicana NCS2 | | | |
| Papaver | ACO90258.1 | S | 144 |
| bracteatum | | | |
| Papaver | AAX56304.1 | S | 181 |
| somniferum NCS2 | | | |
| Tinospora | AKH61504.1 | G | 142 |
| cordifolia NCS | | | |
| Papaver | ACO90257.2 | S | 313 |
| bracteatum | | | |
| Papaver | AKF41585.1 | S | 140 |
| somniferum NCS | | | |
| Expression vector | AKH61498.1 | S | 143 |
| pWCD2338 | | | |
| Eschscholzia | ACO90254.1 | G | 144 |
| californica | | | |
| Papaver | ACI45396.1 | S | 181 |
| somniferum | | | |
| Papaver | AAX56303.1 | S | 181 |
| somniferum NCS1 | | | |
| Papaver | AAX56075.1 | D | 142 |
| somniferum | | | |
| Setaria | XP_004956360.1 | A | 142 |
| italica | | | |
| Eschscholzia | ACO90255.1 | G | 143 |
| californica | | | |
| Papaver | AAX56076.1 | G | 144 |
| somniferum | | | |
| Nelumbo | XP_010247383.1 | G | 143 |
| nucifera NCS1 | | | |
| Nelumbo | XP_010274281.1 | G | 142 |
| nucifera NCS3 | | | |
| Nelumbo | XP_010247387.1 | Q | 140 |
| nucifera NCS4 | | | |
| Nelumbo | XP_010247382.1 | E | 142 |
| nucifera NCS5 | | | |
| (S-norcoclaurine | | | |
| synthase 2-like) | | | |
| Nelumbo | AND61512.1 | W | 143 |
| nucifera NCS7 | | | |
| (S-norcoclaurine | | | |
| synthase 1-like) | | | |

### Concatemer

A concatemer is a multimeric molecule of DNA formed by multiple Bet v I allergen domains (PF00407) arranged linearly in the same head-to-tail orientation. NCSs have been reported to also occur in 2 or more fused copies.

Based on protein sequence, C. japonica NCS (SEQ ID NO: 1) is classified into the PFAM family Bet v I allergen domains (PF00407).

In case a transcript contains multiple Bet v I allergen domains (PF00407), instead of directly aligning SEQ ID NO: 1 with such a concatemer, one should first identify those domains in the translated sequence with for example PFAM and then align the individual domains and not the complete sequence to SEQ 1.

NCS is a relatively inefficient enzyme as observed by low product formation in heterologous genetically engineered systems and by measuring catalytic activity biochemically. Its catalytic efficiency of 1 mM−1s−1 has been reported to be more that 100-fold lower than the median calculated over all enzymes and the high Km for dopamine indicates that high substrate concentrations may be required for significant product formation. In Papaveraceae NCS occurs as fusions of two to four repeated domains with confirmed activity. These fusions may constitute a mechanism to increase efficiency of the enzyme.

Thus, the invention also relates to concatemers of the NCS amino acid sequences described herein.

### NCS Species Origin

The norcoclaurine synthases from Thalictrum flavum is best studied; both biochemical and structural data are available for this enzyme. Low activity is also indicated by the difference in norcoclaurine concentration versus dopamine concentration in yeast strains producing norcoclaurine, being the molar norcoclaurine concentration lower by a factor of up to 100-fold. DeLoache et al, used an NCS from P. somniferum (GenBank accession code KP262411), which was selected after screening four NCS variants with homology to a Thalictrum flavum NCS (GenBank accession code ACO90248.1) for activity in S. cerevisiae.

The inventors used a synthase derived from Coptis japonica.

### Increased Activity

The inventors constructed a protein model of NCS from Coptis japonica using the available structural template from Thalictrum flavum. Correspondingly, an active site model with accommodated relevant substrates and intermediates was generated.

The model was used to design mutations, which were likely to alter enzymatic activity. Alongside, further techniques which selected mutations based on phylogeny and occurrence were utilized. This led to the identification of mutation sites across the protein which were predicted to increase activity and/or substrate selectivity.

Accordingly, rounds of 30-90 mutations were constructed and a yeast strain was transformed with these constructs. The resulting strains were grown for 72 h in 96-well deep well plates at 30° C. in SC-HIS medium with 0.6 mM ammonium sulfate, 1 mM additional tyrosine and 1.5 mg/ml dopamine.

Samples of the cell culture supernatant were subjected to LC/MS analysis for quantification of norcoclaurine, and under these assay conditions several mutants with increased activity of up to 2-fold compared to wild type were identified (See also FIG. 4).

Thus, in one or more exemplary embodiments, the invention relates to a norcoclaurine synthase having an increased catalysation when compared to the wild type synthase of the condensation of 4-HPAA and dopamine to (S)-norcoclaurine and/or 3,4-DhPAA and dopamine to (S)-norlaudanosoline.

The increased catalysation may manifest in increased product formation of cell cultures after 72 hours cultivation time in the assay conditions described above compared to the norcoclaurine production obtained from a cell comprising SEQ ID NO: 1.

Increased catalytic activity can also be shown in enzymatic assays using a lysate from a bacterial of fungal host adding the substrates 4-HPAA or 3,4-DHPAA in combination with dopamine and measure the formation of the products (S)-norcoclaurine or (S)-norlaudanosoline, respectively. In another assay system the heterologously expressed NCS can be purified from a host and used in an in vitro reaction in a suitable buffer system and with favourable salt concentrations together with the substrates. The product can be measured by a suitable LC/MS method.

### Nucleic Acids

In one or more exemplary embodiments, the invention relates to a nucleic acid according to the invention, wherein the nucleic acid sequence is at least 40% identical to the nucleic acid sequence given in SEQ ID No: 2 or SEQ ID No: 4.

As disclosed herein, at least 40% identical to the nucleic acid sequence given in SEQ ID No: 2 or SEQ ID No: 4 relates to at least 45% identical, at least 50% identical, at least 55% identical, at least 60% identical, at least 65% identical, at least 70% identical, at least 75% identical, at least 80% identical, at least 85% identical, at least 90% identical, at least 91% identical, at least 92% identical, at least 93% identical, at least 94% identical, at least 95% identical, at least 96% identical, at least 97% identical, at least 98% identical, at least 99% identical to the nucleic acid sequence given in SEQ ID No: 2 or SEQ ID No: 4.

One or more exemplary embodiments of the invention relates to the nucleic acids described herein encoding the norcoclaurine synthases with the proviso that wild type sequence per se as disclosed in SEQ ID NO: 2 or SEQ ID No: 4 is not included.

When evaluating norcocalurine titers of the cultures after the 72 hours' cultivation time as described herein, the inventors found codon optimized NCS from the flower Coptis japonica is the most active enzyme, see FIG. 3.

In one or more exemplary embodiments, the invention relates to a nucleic acid as described above, wherein the nucleic acid is codon optimized for S. cerevisiae.

In one or more exemplary embodiments, the codon optimized nucleic acid sequence is at least 80% identical to the nucleic acid sequence given in SEQ ID No: 3 or SEQ ID No: 5, such as 81% identical, 82% identical, 83% identical, 84% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, or 100 identical to the nucleic acid sequence given in SEQ ID No: 3 or SEQ ID No: 5.

The skilled person would understand that any feature and/or embodiment discussed above in connections with the amino acids according to the invention apply by analogy to the nucleic acids described herein, especially the necessary means for making the site-specific mutations.

The skilled person would understand that the nucleic acids of the invention may be recombinant.

### Heterologous Host Cell

At least one of the genes mentioned herein can be a recombinant gene, the particular recombinant gene(s) depending on the species or strain selected for use. Additional genes or biosynthetic modules can be included in order to increase compound yield, improve efficiency with which energy and carbon sources are used to produce the target compounds mentioned herein, and/or to enhance productivity from the cell culture or plant, in a engineered biosynthetic pathway.

The invention also relates to heterologous host cell comprising the nucleic acids described herein. Thus, in one embodiment, the invention relates to a heterologous host cell comprising a nucleic acid according to the invention, wherein the nucleic acid is recombinant.

In some embodiments of the invention, the recombinant host comprises a yeast cell, a plant cell, a mammalian cell, an insect cell, a fungal cell, an algal cell, a cyanobacteria or a bacterial cell.

In some embodiments of the invention, microorganisms can include, but are not limited to, S. cerevisiae and E. coli. The constructed and genetically engineered microorganisms provided by the invention can be cultivated using conventional fermentation processes, including, inter alia, chemostat, batch, fed-batch cultivations, continuous perfusion fermentation, and continuous perfusion cell culture.

A number of prokaryotes and eukaryotes are suitable for use in constructing the recombinant microorganisms described herein, e.g., gram-negative bacteria, yeast and fungi. A species and strain selected for use as a strain for production of the compounds described herein is first analysed to determine which production genes are endogenous to the strain and which genes are not present. Genes for which an endogenous counterpart is not present in the strain are assembled in one or more recombinant constructs, which are then transformed into the strain in order to supply the missing function(s).

Exemplary prokaryotic and eukaryotic species are described in more detail below. However, it will be appreciated that other species can be suitable. For example, suitable species can be in a genus selected from the group consisting of Agaricus, Aspergillus, Bacillus, Candida, Corynebacterium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces and Yarrowia. Exemplary species from such genera include Lentinus tigrinus, Laetiporus sulphureus, Phanerochaete chrysosporium, Pichia pastoris, Physcomitrella patens, Rhodoturula glutinis 32, Rhodoturula mucilaginosa, Phaffia rhodozyma UBV-AX, Xanthophyllomyces dendrorhous, Fusarium fujikuroil Gibberella fujikuroi, Candida utilis and Yarrowia lipolytics. In some aspects, a microorganism can be an Ascomycete such as Gibberella fujikuroi, Kluyveromyces lactis, Schizosaccharomyces pombe, Aspergillus niger, or Saccharomyces cerevisiae.

In some embodiments, a microorganism can be a prokaryote such as Escherichia coli, Rhodobacter sphaeroides, or Rhodobacter capsulatus. It will be appreciated that certain microorganisms can be used to screen and test genes of interest in a high throughput manner, while other microorganisms with desired productivity or growth characteristics can be used for large-scale production of the compounds described herein.

### Yeast

In some embodiments, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.

In some embodiments, the yeast cell is a Saccharomycete.

In some embodiments, the yeast cell is a Saccharomyces cerevisiae cell.

### Saccharomyces cerevisiae

Saccharomyces cerevisiae is a widely-used organism in synthetic biology and can be used as the recombinant microorganism platform. There are libraries of mutants, plasmids, detailed computer models of metabolism and other information available for S. cerevisiae, allowing for rational design of various modules to enhance product yield. Methods are known for making recombinant microorganisms.

The genes described herein can be expressed in yeast using any of a number of known promoters.

In some embodiments, auxotrophic markers for cloning include, but are not limited to, HIS3, URA3, TRP1, LEU2, LYS2, ADE2, and GAL, which allow for selection of recombinant strains with an inserted gene of interest. Auxotrophic markers can be optionally removed from the yeast genome using methods not limited to Cre-Lox recombination or negative selection with 5-fluoroorotic acid (5-FOA). In other aspects, antibiotic resistance, such as kanamycin, can be used as selection marker for construction of recombinant strains.

### Algal

In some embodiments, the algal cell is a cell from Blakeslea trispora, Dunaliella salina, Haematococcus pluvialis, Chlorella sp., Undaria pinnatifida, Sargassum, Laminaria japonica, Scenedesmus almeriensis species.

### Cyanobacerial Cell

In some embodiments, the cyanobacerial cell is a cell from Phormidium laminosum, Microcystis sp., Synechococcus sp., Pantoea sp., Flavobacterium sp.

### E. coli

E. coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

### Arxula adeninivorans (Blastobotrys adeninivorans)

Arxula adeninivorans is dimorphic yeast (it grows as budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

### Yarrowia lipolytica

Yarrowia lipolytica is dimorphic yeast (see Arxula adeninivorans) and belongs to the family Hemiascomycetes. The entire genome of Yarrowia lipolytica is known. Yarrowia species is aerobic and considered to be non-pathogenic. Yarrowia is efficient in using hydrophobic substrates (e.g. alkanes, fatty acids, oils) and can grow on sugars. It has a high potential for industrial applications and is an oleaginous microorganism.

Yarrowia lipolyptica can accumulate lipid content to approximately 40% of its dry cell weight and is a model organism for lipid accumulation and remobilization. See e.g., Nicaud, 2012, Yeast 29(10) :409-18; Beopoulos et al., 2009, Biochimie 91(6) : 692-6; Bankar et al., 2009, Appl Microbiol Biotechnol. 84(5) :847-65.

### Rhodotorula sp.

Rhodotorula is unicellular, pigmented yeast. The oleaginous red yeast, Rhodotorula glutinis, has been shown to produce lipids and carotenoids from crude glycerol (Saenge et al., 2011, Process Biochemistry 46(I) : 210-8). Rhodotorula toruloides strains have been shown to be an efficient fed-batch fermentation system for improved biomass and lipid productivity (Li et al., 2007, Enzyme and Microbial Technology 41:312-7).

### Rhodosporidium toruloides

Rhodosporidium toruloides is oleaginous yeast and useful for engineering lipid-production pathways (See e.g. Zhu et al., 2013, Nature Commun. 3:1112; Ageitos et al., 2011, Applied Microbiology and Biotechnology 90(4):1219-27).

### Candida boidinii

Candida boidinii is methylotrophic yeast (it can grow on methanol). Like other methylotrophic species such as Hansenula polymorpha and Pichia pastoris, it provides an excellent platform for producing heterologous proteins. Yields in a multigram range of a secreted foreign protein have been reported. A computational method, IPRO, recently predicted mutations that experimentally switched the cofactor specificity of Candida boidinii xylose reductase from NADPH to NADH. See, e.g., Mattanovich et al., 2012, Methods Mol Biol. 824: 329-58; Khoury et al., 2009, Protein Sci. 18(10):2125-38.

### Hansenula polymorpha (Pichia angusta)

Hansenula polymorpha is methylotrophic yeast (see Candida boidinii). It can furthermore grow on a wide range of other substrates; it is thermo-tolerant and can assimilate nitrate (see also Kluyveromyces lactis). It has been applied to producing hepatitis B vaccines, insulin and interferon alpha-2a for the treatment of hepatitis C, furthermore to a range of technical enzymes. See, e.g., Xu et al., 2014, Virol Sin. 29(6):403-9.

### Kluyveromyces lactis

Kluyveromyces lactis is yeast regularly applied to the production of kefir. It can grow on several sugars, most importantly on lactose which is present in milk and whey. It has successfully been applied among others for producing chymosin (an enzyme that is usually present in the stomach of calves) for producing cheese. Production takes place in fermenters on a 40,000 L scale. See, e.g., van Ooyen et al., 2006, FEMS Yeast Res. 6(3):381-92.

### Pichia pastoris

Pichia pastoris is methylotrophic yeast (see Candida boidinii and Hansenula polymorpha). It provides an efficient platform for producing foreign proteins. Platform elements are available as a kit and it is worldwide used in academia for producing proteins. Strains have been engineered that can produce complex human N-glycan (yeast glycans are similar but not identical to those found in humans). See, e.g., Piirainen et ai, 2014, N Biotechnol. 31(6):532-7.

### Physcomitrella spp.

Physcomitrella mosses, when grown in suspension culture, have characteristics similar to yeast or other fungal cultures. These genera can be used for producing plant secondary metabolites, which can be difficult to produce in other types of cells.

Methods for the preparation of a (S)-norcoclaurine and/or (S)-norlaudanosoline One further aspect of the invention relates to methods for the preparation of a (S)-norcoclaurine and/or (S)-norlaudanosoline compound. Increased catalytic activity can also be shown in enzymatic assays using a lysate from a bacterial of fungal host adding the substrates 4-HPAA or 3,4-DHPAA in combination with dopamine and measure the formation of the products (S)-norcoclaurine or (S)-norlaudanosoline, respectively.

In another assay system, the heterologously expressed NCS can be purified from a host and used in an in vitro reaction in a suitable buffer system and with favourable salt concentrations together with the substrates. The product can be measured by a suitable LC/MS method.

In one or more exemplary embodiments, the method for the preparation of a (S)-norcoclaurine and/or (S)-norlaudanosoline compound comprise the steps of
providing a recombinant host cell capable of catalysation of the condensation of 4-HPAA and dopamine to (S)-norcoclaurine and/or 3,4-DhPAA and dopamine to (S)-norlaudanosoline,
culturing the cells under conditions promoting said catalysation, and optionally
isolating the (S)-norcoclaurine and/or (S)-norlaudanosoline.

In one or more exemplary embodiments, the invention relates to a method for the preparation of a (S)-norcoclaurine and/or (S)-norlaudanosoline compound, the method comprising the steps of:
providing recombinant yeast cells, preferably Saccharomyces cerevisiae capable of catalysation of the condensation of 4-HPAA and dopamine to (S)-norcoclaurine and/or 3,4-DhPAA and dopamine to (S)-norlaudanosoline,
culturing the cells under conditions promoting said catalysation, and optionally
isolating the (S)-norcoclaurine and/or (S)-norlaudanosoline.

In one or more exemplary embodiments, the (S)-norcoclaurine and/or (S)-norlaudanosoline are intermediates in a benzylisoquinoline (BIA) biosynthesis.

One or more exemplary embodiments of the invention relates to a method for the preparation of a (S)-norcoclaurine and/or (S)-norlaudanosoline compound, comprising contacting compound 4-HPAA and dopamine and/or 3,4-DhPAA and dopamine with a recombinant norcoclaurine synthase according to the invention capable of catalysation of the condensation of 4-HPAA and dopamine to (S)-norcoclaurine and/or 3,4-DhPAA and dopamine to (S)-norlaudanosoline.

One or more exemplary embodiments of the invention relates to a method according to method for the preparation of a (S)-norcoclaurine and/or (S)-norlaudanosoline compound as described above, further comprising cultivating a recombinant host cell of the invention in a culture medium in presence of 4-HPAA and dopamine and/or 3,4-DhPAA and dopamine, under conditions in which the one or more recombinant genes encoding the recombinant mutated norcoclaurine synthase(s) according to the invention is/are expressed in presence of 4-HPAA and dopamine and/or 3,4-DhPAA and dopamine.

In one or more exemplary embodiments, said method is performed in vivo.

In one or more exemplary embodiments, said method is performed in vitro.

TABLE 2
Tested Norcoclaurine synthases

| | Protein ID | Nucleotide |
|---|---|---|
| NCS homolog | (Uniprot or NCBI) | ID |
| Thalictrum | AAR22502.1 | AY376412 |
| flavum NCS | | |
| Thalictrum | AAR22502.1 | codon optimized |
| flavum NCS | | |
| Thalictrum | Based on | codon optimized |
| flavum NCS | AAR22502.1 | |
| GS (Δ1-19) | | |
| Thalictrum | Based on | codon optimized |
| flavum NCS | AAR22502.1 | |
| (Δ1-19) | | |
| Thalictrum | Based on | codon optimized |
| flavum NCS GS | AAR22502.1 | |
| Coptis | BAF45338.2 | AB267399.2 |
| japonica NCS | | |
| Coptis | BAF45338.2 | codon optimized |
| japonica NCS | | |
| Coptis | BAF45337.1 | codon optimized |
| japonica | | |
| NCS Long | | |
| Nelumbo | A0A191T7N6 | codon optimized |
| nucifera NCS1 | (XP_010247383.1) | |
| Nelumbo | A0A191T7P4/ | codon optimized |
| nucifera NCS3 | XP_010274281.1 | |
| Nelumbo | A0A1U7Z8P0/ | codon optimized |
| nucifera 4 | XP_010247387.1 | |
| Nelumbo | A0A182C8V5/ | codon optimized |
| nucifera NCS5 | XP_010247382.1 | |
| (in database called | | |
| S-norcoclaurine | | |
| synthase 2-like) | | |
| Nelumbo | A0A182C8V6/ | codon optimized |
| nucifera NCS7 | AND61512.1 | |
| (in database called | | |
| S-norcoclaurine | | |
| synthase 1 -like) | | |
| Argemone | ACJ76785.1 | codon optimized |
| mexicana NCS1 | | |
| Argemone | ACJ76787.1 | codon optimized |
| mexicana NCS2 | | |
| Papaver | AKF41585.1 | codon optimized |
| somniferum NCS | | |
| Papaver | AAX56303 | codon optimized |
| somniferum NCS1 | | |
| Papaver | AAX56304.1 | codon optimized |
| somniferum NCS2 | | |
| Sinopodophyllum | A0A097H1C2 | codon optimized |
| hexandrum NCS | | |
| Tinospora | AKH61504 | codon optimized |
| cordifolia NCS | | |

### General

It should be understood that any feature and/or aspect discussed above in connections with the amino acids according to the invention apply by analogy to the nucleotides described herein.

It should be understood that any feature and/or aspect discussed above in connections with the host cells according to the invention apply by analogy to the methods described herein.

All publications, patents and patent applications cited herein are hereby expressly incorporated by reference for all purposes.

Methods well known to those skilled in the art can be used to construct genetic expression constructs and recombinant cells according to this invention. These methods include in vitro recombinant DNA techniques, synthetic techniques, in vivo recombination techniques, and PGR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA).

A number of terms use herein will be defined below:

As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the invention.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

As used herein, the terms “microorganism,” “microorganism host,” “microorganism host cell,” “host cell,” “recombinant host,” “recombinant microorganism host,” and “recombinant host cell” can be used interchangeably. As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into the non-recombinant host. It will be appreciated that typically the genome of a recombinant host described herein is augmented through stable introduction of one or more recombinant genes.

Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. Said recombinant genes are particularly encoded by cDNA.

As used herein, the term “engineered biosynthetic pathway” refers to a biosynthetic pathway that occurs in a recombinant host, as described herein. In some aspects, one or more steps of the biosynthetic pathway do not naturally occur in an unmodified host. In some embodiments, a heterologous version of a gene is introduced into a host that comprises an endogenous version of the gene.

As used herein, the term “endogenous” gene refers to a gene that originates from and is produced or synthesized within a particular organism, tissue, or cell. In some embodiments, the endogenous gene is a yeast transporter. In some embodiments, the transporter is endogenous to S. cerevisiae, including, but not limited to S. cerevisiae strain S288C. In some embodiments, an endogenous yeast transporter gene is overexpressed. As used herein, the term “overexpress” is used to refer to the expression of a gene in an organism at levels higher than the level of gene expression in a wild type organism. See, e.g., Prelich, 2012, Genetics 190 : 841-54. In some embodiments, an endogenous yeast transporter gene is deleted. See, e.g., Giaever & Nislow, 2014, Genetics 197(2) :451-65. As used herein, the terms “deletion,” “deleted,” “knockout,” and “knocked out” can be used interchangabley to refer to an endogenous gene that has been manipulated to no longer be expressed in an organism, including, but not limited to, S. cerevisiae. In some embodiments, a deleted/knocked out gene is a transporter gene or a transcription factor gene that regulates expression of a transporter gene.

As used herein, the terms “heterologous sequence” and “heterologous coding sequence” are used to describe a sequence derived from a species other than the recombinant host or a sequence from the host that has been inserted into the host recombinantly. In some embodiments are wild type sequences inserted to generate and overexpression of the specific gene. The overexpression can come from manipulation of for example the promoter. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

As used herein, the terms “variant” and “mutant” are used to describe a protein sequence that has been modified at one or more amino acids, compared to the wild type sequence of a particular protein.

The following figures and examples are provided below to illustrate the invention.

They are intended to be illustrative and are not to be construed as limiting in any way.

### BRIEF DESCRIPTION OF THE FIGURES

FIG. 1—Condensation

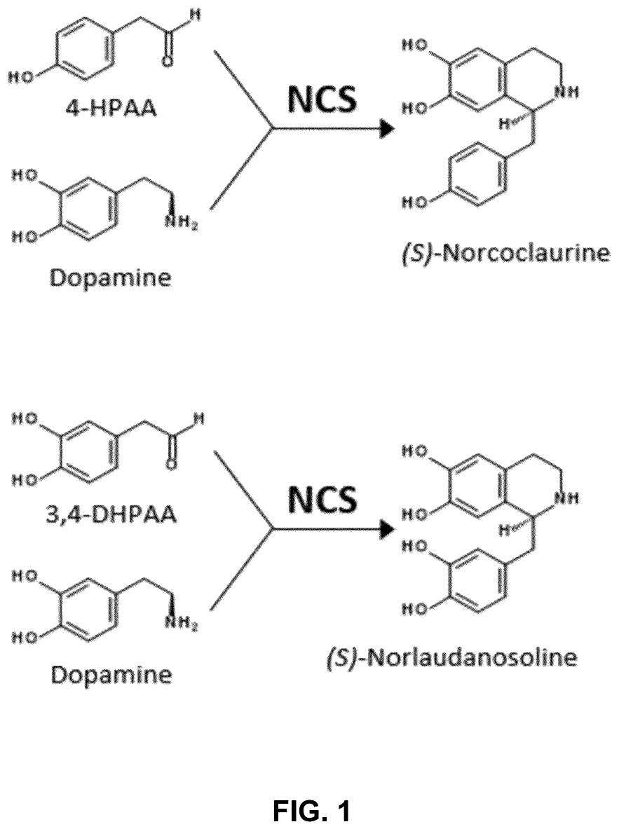

NCS catalyzes the condensation of 4-HPAA and dopamine to (S)-norcoclaurine or 3,4-DHPAA and dopamine to (S)-norlaudanosoline.

FIG. 2—pEV3307 plasmid

Schematic map of pEV3307 plasmid. Vector elements are annotated.

FIG. 3—Norcoclaurine biosynthesis in yeast expressing natural homologs

Screening of NCS homologs from various plant species. Norcoclaurine titers of the cultures after the 72 hours' cultivation time are represented by bars, optical densities of cultures are shown by dots.

FIG. 4—Norcoclaurine biosynthesis in yeast expressing NCS mutants

Screening of variants of norcoclaurine synthase from Coptis japonica. Norcolalurine titers of the cultures after the 72 hours' cultivation time are represented by bars, optical densities of cultures are shown by dots.

FIG. 5—Cj NCS homology model

Cj NCS homology model with active site predicted residues shown (stick form) and reactive substrates shown in space filling format.

FIG. 6—HPAA substrate enhanced interaction with mutation I178D (magenta)

Modelled interaction between mutant I178D (magenta) and HPAA substrate (cyan). According to this model I178D is predicted to directly interact with aldehyde substrates.

FIG. 7—Disruption of signal peptide function of the C. japonica NCS

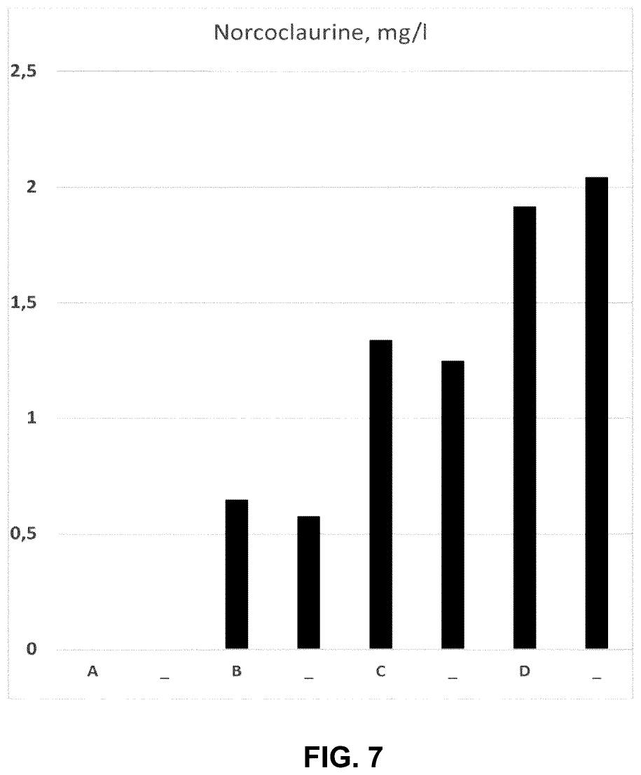

Norcoclaurine production in S. cerevisiae increases dramatically when the first 19 amino acids from the N-terminal of the C. japonica NCS are replaced by a methionine. Duplicate determinations of norcoclaurine production in 4 different S. cerevisiae.

FIG. 8—HDEL (SEQ ID No:22) addition in the C-terminus

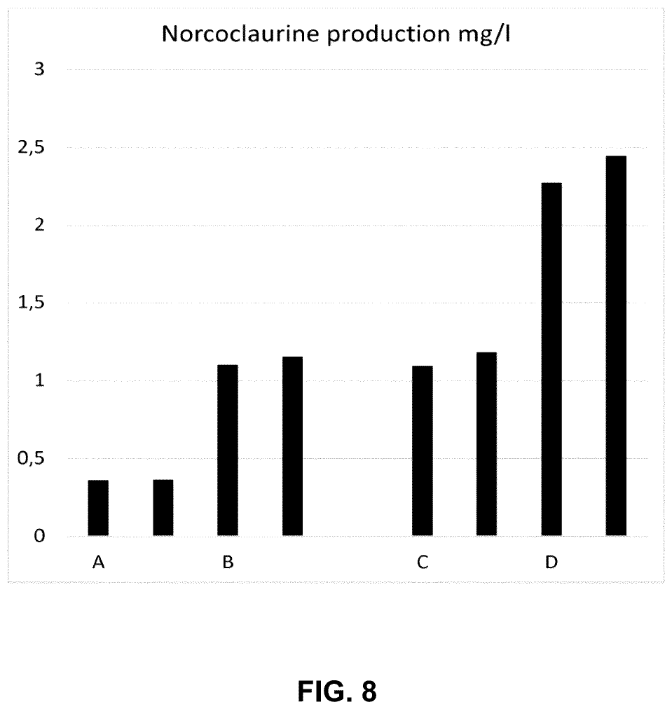

The CjNCS I178D improves the activity of the C. japonica NCS version with the HDEL (SEQ ID No: 22) addition in the C-terminus. Duplicate determinations of norcoclaurine production in 4 different S. cerevisiae strains. A) 11ari1::Aro4^{FBR} CYP76AD1^{W13L, F309L} PpDODC CjNCS1co. B) 11ari1: : Aro4FBR CYP76AD1W13L, F309L PpDODC CjNCS1co/178D C) 11ari1::Aro4^{FBR} CYP76AD1 ^{W13L, F309L} PpDODC CjNCS1co-HDEL. D) 11ari1::Aro4^{FBR} CYP76AD1W^{13L, F309L} PpDODC CjNCS1co^{I178D}-HDEL.

### EXAMPLES

### Example 1—Protein Model of NCS From Coptis japonica—Structural Template

Protein sequences of Coptis japonica (Cj) and Thalictrum flavum (Tf) NCS were aligned using Clustal Omega (Sievers et al., 2011, Mol Syst Biol. 7 : 539) along with other NCS homologs identified using the BLASTP algorithm up to 40% identity.

The sequence of Cj NCS has 63% global protein sequence identity to the sequence of Thalictrum flavum (Tf) NCS, indicating that their three-dimensional protein structures are highly likely to be similar. Therefore, a protein model of Cj norcoclaurine synthase (NCS) was constructed using the atomic co-ordinates of the protein structure of NCS from Thalictrum flavum (Tf) (PDB code 2VQ5; Ilari et al. 2009).

A homology model of Cj NCS was constructed using Homology Model tool in MOE (CCG Inc). A protein modelling software well known and available in the art.

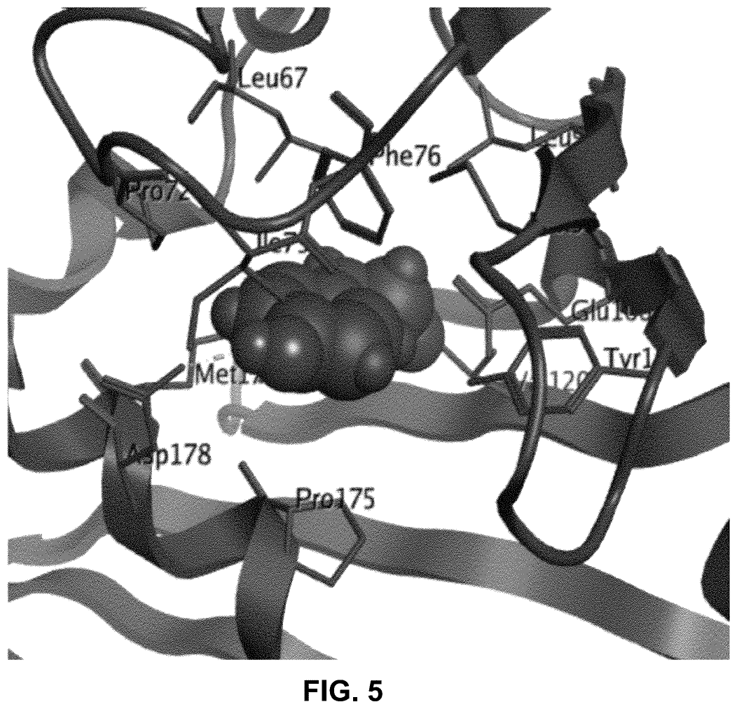

The Cj NCS model allows key residue-specific interactions at the interface between Cj NCS and substrates dopamine and 4-hydroxyphenylacetylaldehyde to be discerned. Final models were constructed with reactive modelled conformations of dopamine and 4-hydroxyphenylacetylaldehyde [FIG. 5]. These protein models provided the basis to select key residues to mutate in order to affect ligand binding potentials.

Obtained gene sequences were codon optimized for expression in Saccharomyces cerevisiae, and their synthesis was performed by Thermofischer Scientific Int.

Synthetized genes were cloned in pEV3307 following the procedure described in Example 3. The mutants showing increased NCS versus the wild type enzyme are summarized in Table 8.

### Example 2—Cj NCS Activity Increase by Enzyme Engineering Approach—Active Site Model->Design Mutations

From the constructed homology model, amino acids were selected for in silico mutation studies using the PROTOSCAN methodology as described by example in WO 2013022881. Briefly, selected amino acids are swapped for each of 19 possible amino acid mutations, the coordinates of which are determined by selection of low energy rotamers before calculating both intra and intermolecular energy of interaction versus wild type residue.

Thus, amino acid mutations can be elucidated, which provide lower binding energy in the context of substrates.

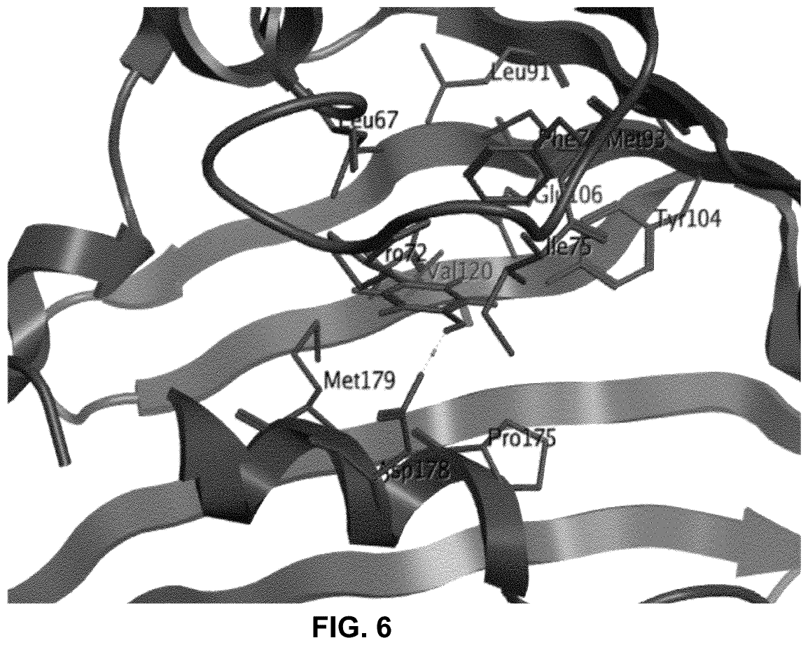

For example, position 178 of NCS contains Isoleucine. PROTOSCAN ranked energies of other mutations at this position and aspartic acid had the lowest energy, because of enhanced interaction with the para hydroxyl group of HPAA substrate as shown in FIG. 6.

The mutation was tested and demonstrated higher activity than the wild type protein. Thus, the in silico screen identified enriching mutation selection versus a random selection of mutants and can effectively sample sequence space in particular, where a substrate binding model is available.

### Example 3—Preparation of Expression Vectors

NCS gene sequences from different organisms (Table 2) were identified and codon optimized for expression in Saccharomyces cerevisiae.

The synthesis of the resulting sequences was performed by Thermofisher Scientific Inc. Gene sequences and related organism of origin are shown in Table 2.

The newly synthesized genes were cloned in the yeast expression plasmid “pEV3307” (pRS313 modified with the insertion of PGK1 and TEF1 promoters, CYC1 and ADH 1 terminators. For basic plasmid details see Mumberg et al. 1995, Yeast vectors for the controlled expression of heterologous proteins in different genetic backgrounds, Gene 156(I):119-22, 1995) carrying the HIS3 auxotrophic marker.

HindIII/SacII restriction sites were utilized for the insertion of the open reading frames between PGK1 promoter and CYC terminator.

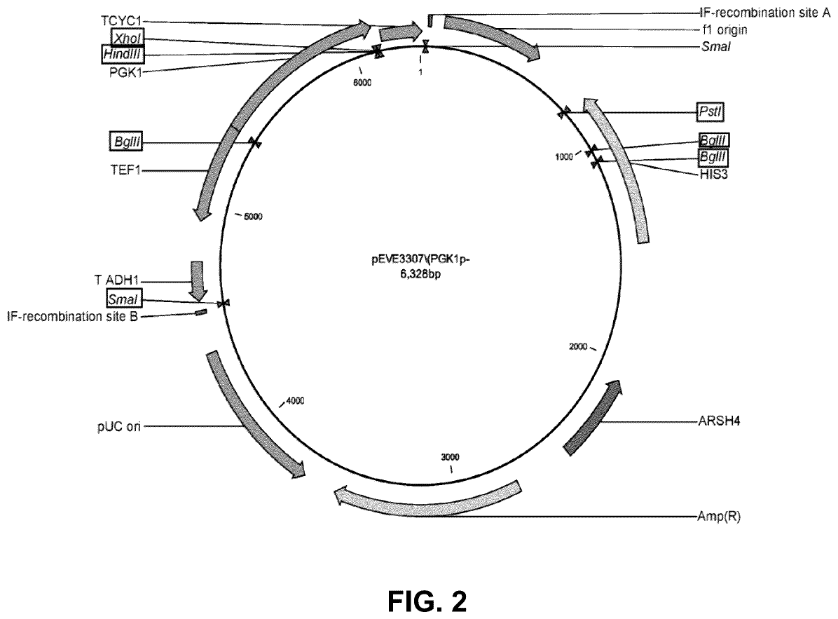

A schematic map of plasmid pEVE3307 with related annotations is shown in FIG. 2.

### Example 4—Strain Construction and Expression of NCS Homologs

EVST25898, a modified version of S288C yeast strain (genotype: MATalpha his3A0 leu2Δ0 ura3A0 aro3Δ::pTEFI-ARO4(K229L)-tCYCI::pPGKI-ARO7(T266L)-tADH1::KICAT5-91Met GAL2 ho MIP1-661Thr SAL1-1 YORWΔ22::npBIO1nt-npBIO6nt) was transformed with the constructs described in Example 1.

Yeast transformation was performed according to conventional methods (see R. D. Gietz and R. Woods, “Yeast Transformation by the LiAc/SS Carrier DNA/PEG Method,” in Yeast Protocol SE—12, vol. 313, W. Xiao, Ed. Humana Press, 2006, pp. 107-120).

The resulting yeast strains were grown in a 96-deep well plate in appropriate medium (standard SC medium without histidine) for 24 h at 30° C. (pre-culture) with 300 rpm shaking. Cells were then inoculated into fresh SC-HIS medium reaching an initial OD600˜0.1. Obtained cultures were incubated at 30° C. for 72 h. After 72 h the cells were harvested and the culture supernatants were analyzed according to the methods reported in Examples 6 and 7.

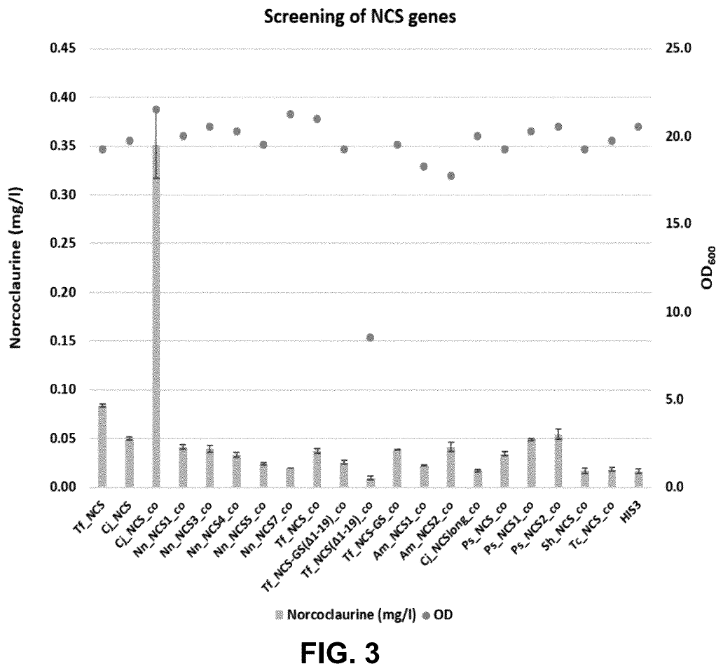

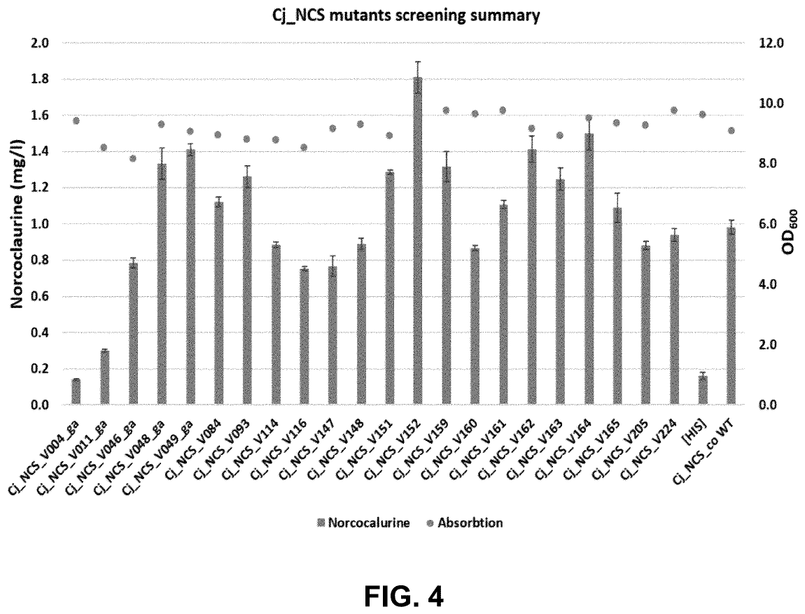

Norcoclaurine was detected in the supernatant of the strain carrying Coptis japonica NCS, at a concentration of ˜0.35 mg/l. Results are reported in the graph in FIG. 3-4.

### Example 5—Screening of Cj_NCS Mutants

Strain EVST25898 was transformed with the constructs obtained in Example 3 (see Example 2 for genotype) in 96 deep well plate format with a commercially available kit (Froze-EZ Yeast transformation II Kit™, Zymo research) according to the manufacturer instructions.

Transformed cells were grown in SC-HIS medium and incubated for 24 h at 30° C. under mild shaking. 20 μl of the transformation cultures were used to inoculate a pre-culture in SC-HIS medium (in 96-well format) and the resulting plate was incubated for 24 h at 30° C. 5 μl of pre-cultures were inoculated in fresh SC-HIS standard medium, or with modified composition (0.6 mM ammonium sulfate, 1.4 mM tyrosine, 1.5 mg/mg dopamine) and incubated for 72 h.

Analysis of culture supernatants was performed according to the method reported in Examples 6 and 7.

A selection of mutants, including the ones showing increased norcoclaurine production versus the control strain after the first screening, were repeated for experimental confirmation and the results of the analysis are shown in FIG. 4.

Variants V48, V49, V93, V151, V152, V240, V241, V242, V246, V247, V249, V253, V261 and V273 showed increased norcoclaurine production up to about 1.8-fold.

### Example 6—Norcoclaurine Chiral Analysis

Norcoclaurine HCl (also called higenamine, Carbosynth) solution (1 g/L) was prepared in water. A series of calibration solutions at 4 mg/L, 2 mg/L, 1 mg/L, 0.5 mg/L, 0.25 mg/L, 0.125mg/L, 62.5 ug/L and 31.25 ug/L in the culture medium was prepared from this stock solution. Caffeine (Sigma) was added to the samples as internal standard to a concentration of 1mg/L and samples were injected into the UPLC-SQD (Waters).

The LC-MS method was as follows: Mobile Phase A: water+0.1% formic acid; Mobile Phase B: acetonitrile+0.1% formic acid; Column : ORpakCDBS453 (Shodex).

The elution gradient is shown in Table 3 and the LC-MS conditions are given in Table 4.

Table 5 shows the mass spectrometer source and detector parameters. Norcoclaurine and Caffeine were injected analysed in Single lon Monitoring mode with the respective mass over charge ratio (m/z) 227 and 195 corresponding to their protonated ion. (S)-Norcoclaurine eluted at 5.8 min, (R)-Norcoclaurine at 6.1 min and Caffeine at 6.3 min.

TABLE 3
Gradient for chiral separation

| Time (min) | % B |
|---|---|
| 0 | 1 |
| 5 | 35 |
| 8 | 100 |
| 9 | 100 |
| 9.1 | 1 |
| 10 | 1 |

TABLE 4
LC-MS conditions

| | |
|---|---|
| Injection volume | 3 μl |
| Column Temperature | 30° C. ± 5° C. |
| Injection method | Partial loop |
| Flow | 0.4 ml/min |
| Auto sampler temperature | 10° C. ± 2° C. |
| Weak wash | 800 μl water/acetonitrile 8:2 |
| Strong wash | 300 μl MeOH |
| Seal wash | 5 min with water/acetonitrile 9:1 |

TABLE 5
Mass spectrometer source and detector parameters (SQD)

| Source Parameter | Value | |
|---|---|---|
| Ion Source | Electrospray Positive | |
| | Mode (ESI+) | |
| Capillary Voltage | 3.5 | kV |
| Cone Voltage | 20 | V |
| Extractor | 3 | V |
| RF lens | 0.1 | V |
| Source Temperature | 150° | C. |
| Desolvation temperature | 350° | C. |
| LM resolution | 15 | |
| HM resolution | 15 | |
| Ion Energy | 0.5 | eV |
| Mode | MS | |
| LM resolution2 | 15 | |
| HM resolution2 | 15 | |
| Ion Energy2 | 0.5 | |
| API gas | 500 | L/hour |

### Example 7—Norcoclaurine Non-Chiral Analysis

Ig/L Higenamine HCl (Carbosynth) solution was prepared in water. A series of calibration solutions at 4 mg/L, 2 mg/L, 1 mg/L, 0.5 mg/L, 0.25 mg/L, 0.125 mg/L, 62.5 g/L and 31.25g/L in the culture medium was prepared from this stock solution. Caffeine (Sigma) was added as internal standard to a concentration of Img/L and samples were injected into the UPLC-SQD (Waters).

The LC-MS method was as follows: Mobile Phase A: water+0.1% formic acid; Mobile Phase B: acetonitrile+0.1% formic acid; Column: Aquity BEH C18100×2.1 mm (Waters).

The elution gradient is shown in Table 6 and the LC-MS conditions are given in Table 7.

Table 5 shows the mass spectrometer source and detector parameters. Norcoclaurine and Caffeine were injected analysed in Single lon Monitoring mode with the respective mass over charge ratio (m/z) 227 and 195 corresponding to their protonated ion. Retention times of Norcoclaurine and Caffeine were 2.5 min and 3.0 min respectively.

TABLE 6
Gradient for non-chiral separation

| Time (min) | % B |
|---|---|
| 0 | 2 |
| 5 | 30 |
| 5.1 | 100 |
| 5.9 | 100 |
| 6 | 2 |
| 8 | 2 |

TABLE 7
LC-MS conditions

| | |
|---|---|
| Injection volume | 5 μl |
| Column Temperature | 30° C. ± 5° C. |
| Injection method | Partial loop |
| Flow | 0.4 ml/min |
| Auto sampler temperature | 10° C. ± 2° C. |
| Weak wash | 800 μl water/acetonitrile 8:2 |
| Strong wash | 300 μl MeOH |
| Seal wash | 5 min with water/acetonitrile 9:1 |

TABLE 8
Mutations with increased activity

| | | Fold increase |
|---|---|---|
| ID | Mutation(s) | mutant/WT |
| Cj_NCS_V48 | V174G | 1.11 |
| Cj_NCS_V49 | I178A | 1.35 |
| Cj_NCS_V84 | K152R | 1.28 |
| Cj_NCS_V93 | V174E | 1.08 |
| Cj_NCS_V114 | V141I V142I K152R | 1.39 |
| Cj_NCS_V116 | D147N K152R T82V | 1.08 |
| Cj_NCS_V151 | I178S | 1.44 |
| Cj_NCS_V152 | I178D | 2.16 |
| Cj_NCS_V159 | K152R V174E | 1.51 |
| Cj_NCS_V161 | K114E V174E | 1.16 |
| Cj_NCS_V162 | V174G K152R | 1.29 |
| Cj_NCS_V163 | V174G K114E | 1.37 |
| Cj_NCS_V164 | I178A K152R | 1.9 |
| Cj_NCS_V240 | I178N | 1.509 |
| Cj_NCS_V241 | I178Q | 1.767 |
| Cj_NCS_V242 | I178T | 1.111 |
| Cj_NCS_V246 | I178D I75L | 1.469 |
| Cj_NCS_V247 | I178D I75K | 1.859 |
| Cj_NCS_V248 | I178D K152R | 2.002 |
| Cj_NCS_V249 | I178D A77S | 1.567 |
| Cj_NCS_V250 | I178D A77S K152R | 1.761 |
| Cj_NCS_V251 | I178D I75K K152R | 1.77 |
| Cj_NCS_V253 | I178D V174Q | 1.429 |
| Cj_NCS_V258 | I178D V174 K152R | 1.279 |
| Cj_NCS_V261 | A73P A77E | 1.036 |
| Cj_NCS_V263 | I178D K114E K152R | 1.152 |
| Cj_NCS_V267 | I178D V174E Q99K | 1.257 |
| Cj_NCS_V268 | I178D V174Q Q99K | 1.786 |
| Cj_NCS_V269 | I178D V174E Q99R | 1.539 |
| Cj_NCS_V270 | I178D V174Q Q99R | 1.912 |
| Cj_NCS_V271 | I178D V174G K152R A77S | 1.937 |
| Cj_NCS_V273 | A77T I178D | 1.163 |

### Example 8—Effect of Norcoclaurine Synthase Localization on Production of Norcoclaurine in S. cerevisiae

Based on the S. cerevisiae gene integration and expression system developed by Mikkelsen, M D et al (Metab. Eng. 14, Issue 2, 104-111 (2012)), a set of integration/recombination plasmids were designed to simultaneously delete the ORF of the S. cerevisiae gene ARI1 and collectively overexpress:
1) a feedback resistant variant of ARO4, ARO4^{FBR} (Luttic, M. A. H et al Metab. Eng. 10, 141-153 (2008),
2) a Tyrosine hydroxylase based on the Beta vulgaris CYP76AD1 (DeLoache, W. C. et al Nat. Chem. Biol., 11, 465-471 (2015)),
3) the Pseudomonas putida DOPA decarboxylase (PpDODC), and
4) several versions of the Coptis japonica Norcoclaurine synthase (CjNCS).

All genes used for overexpression were codon optimized for expression in S. cerevisiae. The expression cassettes in these integration plasmids were designed to give high expression of the norcoclaurine pathway genes and the following promoters were used: PDC1 promoter (ARO4^{FBR}), TDH3 promoter (CYP76AD1 ^{W13L,F309L}), TEF2 promoter (PpDODC), and the PGK1 promoter for expression of the NCS variants.

A laboratory yeast strain (BY4741) transformed with this combined ARI1.

deletion/norcoclaurine biosynthesis gene overexpression system gives rise to transformants that show high production of norcoclaurine compared to what was reported in literature (DeLoache, W. C. et al Nat. Chem. Biol., 11, 465-471 (2015)).

Yeast transformants were grown in 96 deep-well plates in 500 μL liquid Synthetic Complete media for 3 days at 30° C. with shaking at 230 rpm in a Kuhner Climo-Shaker ISF1-X. Culture samples for LC-MS were prepared by extraction as follows: 96% ethanol and culture sample were mixed 1:1 and incubated on a heating block at 80° C. for 10 min. After heating cells were pelleted in an Eppendorff table top centrifuge by centrifugation and the supernatant was then transferred to a new tube and diluted 1:5 in water.

### Example 9—Coptis japonica NCS^{I178D}

To test the performance of the NCS^{I178D} version and the NCS^{I178D} version with HDEL (SEQ ID No: 22) amino acids added in the C-terminus in this system, new plasmids and yeast strains were constructed.

Again the system described in Example 8 was applied, i.e. deletion of the ORF of ARI1 with simultaneous overexpression of ARO4^{FBR}, CYP76AD1 ^{W13L, F309L}, PpDODC and the various versions of the C. japonica NCS.

### Example 10—LC-MS Protocol

A Norcoclaurine stock solution was prepared in DMSO at a concentration of 10 mM. Standard solutions were prepared at concentrations of 4 uM, 2 uM, 1 uM, 500 nM, 200 nM, 100 nM, 50 nM, 20 nM and 10 nM from the stock solution. Samples were injected into the Agilent 1290 UPLC coupled to an Ultivo Triple Quadrupole. The LC-MS method was as follows: Mobile Phase A. H2O+0.1% Formic acid; Mobile Phase B: Acetonitrile +0.1% Formic acid; Column: Phenomenex Kinetex 1.7 um XB-C18 IOOA, 2.1×100 mm. The elution gradient is shown in Table X and the LC-MS conditions are given in Table X. Table X shows the mass spectrometer source and detector parameters and Table X shows the target compound, retention time, parent ion, transition ions (MRM) as well as dwell time, fragmentor voltag and collision energy used.

TABLE 9
Gradient for LC-MS

| Time (min) | % B |
|---|---|
| 0 | 2 |
| 0.30 | 2 |
| 3.00 | 25 |
| 3.40 | 100 |
| 3.90 | 100 |
| 4 | 2 |
| 5 | 2 |

TABLE 10
LC-MS conditions

| Parameter | Value |
|---|---|
| Injection volume | 2 μl |
| Column Temperature | 30° C. ± 4° C. |
| Injection method | Flow through needle |
| Flow | 0.4 ml/min |
| Auto sampler temperature | 10° C. ± 2° C. |
| Reconditioning wash | 2% Acetonitrile (in H₂O), 5 sec |
| Weak wash | 20% Methanol (in H₂O), 5 sec |
| Strong wash | 30% Acetonitrile, 30% Methanol, |
| | 30% 2-Propanol, 10% H₂O, 10 sec |
| Seal wash | 20% 2-Propanol (in H₂O) |

TABLE 11
Mass spectrometer source and detector
parameters (Ultivo Triple Quadrupole)

| Parameter | Value | |
|---|---|---|
| Ion Source | Electrospray Positive | |
| | Mode (ESI+) | |
| Capillary Voltage | 3.5 | kV |
| Nozzle Voltage | 500 | V |
| Source Gas Temperature | 290° | C. |
| Source Gas Flow | 12 | L/min |
| Source Sheath Gas Temperature | 380° | C. |
| Source Sheath Gas Flow | 12 | L/min |
| Nebulizer | 30 | psi |

| | |
|---|---|
| Mode | MS/MS |
| Collision | See Table 4 |

TABLE 12
Multiple reaction monitoring targets and conditions (ESI+)

| | Retention | Parent | Daughter | Dwell | Fragment | Collision |
|---|---|---|---|---|---|---|
| Target | time | ion | ion | time | or voltage | energy |
| compound | (min) | (m/z) | (m/z) | (ms) | (V) | (V) |
| Norcoclaurine | 2.29 | 272 | 255 | 200 | 110 | 5 |

### REFERENCES

Bonamore, A., Barba, M., Botta, B., Boffi, A. and Macone, A., 2010. Norcoclaurine synthase: mechanism of an enantioselective pictet-spengler catalyzing enzyme. Molecules, 15(4), pp.2070-2078
DeLoache, W. C. et al. An enzyme-coupled biosensor enables (S)-reticuline production in yeast from glucose. Nat. Chem. Biol. 11, 465-471 (2015)
Galanie S, Thodey K, Trenchard IJ, Filsinger Interrante M, Smolke CD., 2015. Complete biosynthesis of opioids in yeast. Science. 2015 Sep. 4; 349(6252) : 1095-100.
Ilari, A., Franceschini, S., Bonamore, A., Arenghi, F., Botta, B., Macone, A., Pasquo, A., Bellucci, L. and Boffi, A., 2009. Structural basis of enzymatic (S)-norcoclaurine biosynthesis. Journal of biological chemistry, 284(2), pp.897-904

1Lichman, B.R., Gershater, M.C., Lamming, E.D., Pesnot, T., Sula, A., Keep, N.H., Hailes, H. C. and Ward, J.M., 2015. ‘Dopamine-first’ mechanism enables the rational engineering of the norcoclaurine synthase aldehyde activity profile. FEBS journal, 282(6), pp.1137-1151
Mumberg D, Muller R, Funk M. Yeast vectors for the controlled expression of heterologous proteins in different genetic backgrounds. Gene. 1995; 156 : 119-122.
Narcross, L., Fossati, E., Bourgeois, L., Dueber, J. E. and Martin, V. J. J. Microbial Factories for the Production of Benzylisoquinoline Alkaloids. Trends Biotechnol. 34, 228-241 (2016)
Mumberg D, Muller R, Funk M. Yeast vectors for the controlled expression of heterologous proteins in different genetic backgrounds. Gene. 156, 119-122 (1995)

### SEQUENCE LISTING FREE TEXT

SEQ ID NO 1: Coptis japonica NCS
SEQ ID NO 2: Coptis japonica NCS nucleotide
SEQ ID NO: 3: NCS 2 nucleotide sequence codon optimized for S. cerevisiae
SEQ ID NO: 4: Coptis japonica NCS wild type loop structure sequence
SEQ ID NO: 5: Coptis japonica NCS wild type helix structure sequence
SEQ ID NO: 6: >CYP76AD1 W13L, F309L
SEQ ID NO: 7: >CYP76AD1 W13L, F309L nucleotide sequence codon optimized for S. cerevisiae
SEQ ID NO: 8: >PpDODC
SEQ ID NO: 9: >PpDODC nucleotide sequence codon optimized for S. cerevisiae
SEQ ID NO: 10: >ARO4^{FBR}
SEQ ID NO: 11: >ARO4^{FBR} nucleotide sequence
SEQ ID NO: 12: >CjNCSco
SEQ ID NO: 13: >CjNCSco nucleotide sequence codon optimized for S. cerevisiae
SEQ ID NO: 14: >ΔI 9-CjNCSco
SEQ ID NO: 15: >ΔI 9-CjNCSco nucleotide sequence codon optimized for S. cerevisiae SEQ ID NO: 16: >CjNCSco-HDEL
SEQ ID NO: 17: >CjNCSco-HDEL nucleotide sequence codon optimized for S. cerevisiae
SEQ ID NO: 18: >CjNCSIco^{I178D}
SEQ ID NO: 19: >CjNCSIco^{I178D} nucleotide sequence codon optimized for S. cerevisiae
SEQ ID NO: 20: >CjNCSIco^{I178D}-HDEL
SEQ ID NO: 21: >CjNCSIco^{I178D}-HDEL nucleotide sequence codon optimized for S. cerevisiae

### ITEMS OF THE INVENTION

1. A norcoclaurine synthase comprising a substrate binding amino acid sequence, which is at least 50% similar to the substrate binding amino acid sequence SEQ ID NO: 4 and/or SEQ ID NO: 5, and wherein the substrate binding amino acid sequence(s) comprise one or more mutations increasing the norcoclaurine synthase activity compared to wild type.
2. A norcoclaurine synthase according to item 1, which has the amino acid Proline (P) corresponding to position 2 in the count according to SEQ ID No. 4.
3. A norcoclaurine synthase according to item 1-2, which has the amino acid Leucine (K) or Lysine (L) corresponding to position 4 in the count according to SEQ ID No. 4.
4. A norcoclaurine synthase according to item 1-3, which has the amino acid Serine (S), Threonine (T) or Glutamic Acid (E) corresponding to position 6 in the count according to SEQ ID No. 4.
5. A norcoclaurine synthase according to item 1-4, which has the amino acid Proline (P) corresponding to position 2 and Leucine (K) or Lysine (L) corresponding to position 4 in the count according to SEQ ID No. 4.
6. A norcoclaurine synthase according to item 1-5, which has the amino acid Proline (P) corresponding to position 2 and Serine (S), Threonine (T) or Glutamic Acid (E) corresponding to position 6 in the count according to SEQ ID No. 4.
7. A norcoclaurine synthase according to item 1-6, which has the amino acid Leucine (K) or Lysine (L) corresponding to position 4 and Serine (S), Threonine (T) or Glutamic Acid (E) corresponding to position 6 in the count according to SEQ ID No. 4.
8. A norcoclaurine synthase according to item 1-7, which has the amino acid Proline (P) corresponding to position 2, Leucine (K) or Lysine (L) corresponding to position 4 and Serine (S), Threonine (T) or Glutamic Acid (E) corresponding to position 6 in the count according to SEQ ID No. 4.
9. A norcoclaurine synthase according to item 1-8 which has the amino acid Glycine (G) or Glutamic Acid (E) corresponding to position 2 in the count according to SEQ ID No. 5.
10. A norcoclaurine synthase according to item 1-9, which has the amino acid Alanine (A), Serine (S) or Aspartic Acid (D) corresponding to position 6 in the count according to SEQ ID No. 5.
11. A norcoclaurine synthase according to item 1-10, which has the amino acid Glycine (G) or Glutamic Acid (E) corresponding to position 2 and Alanine (A), Serine (S) or Aspartic Acid (D) corresponding to position 6 in the count according to SEQ ID No. 5.
12. A norcoclaurine synthase according to any of items 1-11, having an increased catalysation when compared to the wild type synthase of the condensation of 4-HPAA and dopamine to (S)-norcoclaurine and/or 3,4-DhPAA and dopamine to (S)-norlaudanosoline.
13. A norcoclaurine synthase according to any of items 1-12, wherein the increased catalysation manifest in increased norcoclaurine production of cell cultures after 72 hours' cultivation time compared to norcoclaurine production obtained from a cell comprising SEQ ID NO 1 (WILD TYPE).
14. A norcoclaurine synthase according to any of items 1-13, wherein the synthase is derived from Coptis japonica.
15. A nucleic acid encoding a norcoclaurine synthase according to any of items 1-14.
16. A nucleic acid according to item 15, wherein the nucleic acid sequence is at least 40% identical to the nucleic acid sequence given in SEQ ID No. 2 (DNA).
17. A nucleic acid according to items 15-16, wherein the nucleic acid is codon optimized for S. cerevisiae.
18. A nucleic acid according to items 15-16, wherein the nucleic acid sequence is at least 80% identical to the nucleic acid sequence given in SEQ ID No. 3.
19. A heterologous host cell comprising a nucleic acid according to any one of items 15-18, wherein the nucleic acid is recombinant.
20. The heterologous host cell according to item 19, wherein the cell is a yeast cell, a plant cell, a mammalian cell, an insect cell, a fungal cell, a bacterial cell, an algal cell, or a cyanobacterial cell.
21. The heterologous host cell according to any one of items 19-20, wherein the cell is a yeast cell selected from the group consisting of Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.
22. The heterologous host cell according to any one of items 19-21, wherein the host cell is a Saccharomyces.
23. The heterologous host cell according to any one of items 19-22, wherein the host cell is a yeast cell is Saccharomyces cerevisiae cell.
24. A method for the preparation of a (S)-norcoclaurine and/or (S)-norlaudanosoline compound, the method comprising the steps of:
   providing a recombinant host cell according to any of items 19-23, wherein the host cell is capable of catalysation of the condensation of 4-HPAA and dopamine to (S)-norcoclaurine and/or 3,4-DhPAA and dopamine to (S)-norlaudanosoline,
   culturing the cell under conditions promoting said catalysation, and optionally
   isolating the (S)-norcoclaurine and/or (S)-norlaudanosoline.
25. A method for the preparation of a (S)-norcoclaurine and/or (S)-norlaudanosoline compound, comprising contacting compound 4-HPAA and dopamine and/or 3,4-DhPAA and dopamine with a recombinant norcoclaurine synthase according to any of items 1-14, wherein the recombinant norcoclaurine synthase is capable of catalysation of the condensation of 4-HPAA and dopamine to (S)-norcoclaurine and/or 3,4-DhPAA and dopamine to (S)-norlaudanosoline.
26. The method according to item 25, further comprising cultivating a recombinant host cell of any one of items 19-23 in a culture medium in the presence of 4-HPAA and dopamine and/or 3,4-DhPAA and dopamine, under conditions in which the one or more recombinant genes encoding the recombinant norcoclaurine synthase according to any of items 1-14 is/are expressed in presence of 4-HPAA and dopamine and/or 3,4-DhPAA and dopamine.
27. The method according to item 25-26, which is performed in vitro.
28. A norcoclaurine synthase comprising a substrate binding amino acid sequence, which is at least 20% identical to the substrate binding amino acid sequence SEQ ID NO: 4 and/or SEQ ID NO: 5, and wherein the substrate binding amino acid sequence(s) comprise one or more mutations increasing the norcoclaurine synthase activity compared to wild type.
29. A norcoclaurine synthase according to item 1 or 28, which has the amino acid Proline (P) corresponding to position 2, Leucine (K) or Lysine (L) corresponding to position 4, and/or Serine (S), Threonine (T) or Glutamic Acid (E) corresponding to position 6 in the count according to SEQ ID No: 4.
30. A norcoclaurine synthase according to item 1 or any of 28-29, which has the amino acid Glycine (G) or Glutamic Acid (E) corresponding to position 2, and/or Alanine (A), Serine (S) or Aspartic Acid (D) corresponding to position 6 in the count according to SEQ ID No: 5.
31. A norcoclaurine synthase according to any of items 1 or any of 28-30, having an increased catalysation when compared to the wild type synthase of the condensation of 4-HPAA and dopamine to (S)-norcoclaurine and/or 3,4-DhPAA and dopamine to (S)-norlaudanosoline.
32. A norcoclaurine synthase according to any of items 1 or any of 28-31, which has the amino acid Proline (P) corresponding to position 2 in the count according to SEQ ID No: 4.
33. A norcoclaurine synthase according to item 1 or any of 28-32, which has the amino acid Leucine (K) or Lysine (L) corresponding to position 4 in the count according to SEQ ID No: 4.
34. A norcoclaurine synthase according to item 1 or any of 28-33, which has the amino acid Serine (S), Threonine (T) or Glutamic Acid (E) corresponding to position 6 in the count according to SEQ ID No: 4.
35. A norcoclaurine synthase according to item 1 or any of 28-34, which has the amino acid Proline (P) corresponding to position 2 and Leucine (K) or Lysine (L) corresponding to position 4 in the count according to SEQ ID No: 4.
36. A norcoclaurine synthase according to item 1 or any of 28-35, which has the amino acid Proline (P) corresponding to position 2 and Serine (S), Threonine (T) or Glutamic Acid (E) corresponding to position 6 in the count according to SEQ ID No: 4.
37. A norcoclaurine synthase according to item 1 or any of 28-36, which has the amino acid Leucine (K) or Lysine (L) corresponding to position 4 and Serine (S), Threonine (T) or Glutamic Acid (E) corresponding to position 6 in the count according to SEQ ID No: 4.
38. A norcoclaurine synthase according to item 1 or any of 28-37, which has the amino acid Proline (P) corresponding to position 2, Leucine (K) or Lysine (L) corresponding to position 4 and Serine (S), Threonine (T) or Glutamic Acid (E) corresponding to position 6 in the count according to SEQ ID No: 4
39. A nucleic acid encoding a norcoclaurine synthase according to any of items 1 or any of 28-38, wherein the nucleic acid sequence is at least 40% identical to the nucleic acid sequence given in SEQ ID No: 4.
40. A heterologous host cell comprising a nucleic acid according to any one of items 1 or any of 28-39, wherein the nucleic acid is recombinant.
41. The heterologous host cell according to item 40, wherein the host cell is a Saccharomyces cerevisiae.
42. A norcoclaurine synthase comprising an amino acid sequence which is at least 40% identical to the amino acid sequence given in SEQ ID No. 1, and which has one or more site-specific mutations corresponding to position 73, 75, 77, 82, 99, 114, 141, 142, 147, 152, 174 and/or 178 of SEQ ID No. 1.
43. A norcoclaurine synthase according to item 42, wherein the site-specific mutation is selected from the group consisting of mutations corresponding A73P, I75L, I75K, A77S, A77E, A77T, T82V, Q99K, Q99R, K114E, V1411, V1421, D147N, K152R, V174E, V174G, V174Q, V174E, I178A, I178S, I178D, I178N, I178Q, and I178T of SEQ ID No.
44. A norcoclaurine synthase according to any of items 42-43, having an increased catalysation when compared to the wild type synthase of the condensation of 4-HPAA and dopamine to (S)-norcoclaurine and/or 3,4-DhPAA and dopamine to (S)-norlaudanosoline.
45. A norcoclaurine synthase according to any of items 42-44, wherein the synthase is derived from Coptis japonica.
46. A nucleic acid encoding a norcoclaurine synthase according to any of claims items 42-45.
47. A nucleic acid according to claim 46, wherein the nucleic acid sequence is at least 40% identical to the nucleic acid sequence given in SEQ ID No. 2.
48. A nucleic acid according to claims items 46-47, wherein the nucleic acid is codon optimized for S. cerevisiae.
49. A nucleic acid according to items 46-47, wherein the nucleic acid sequence is at least 60% identical to the nucleic acid sequence given in SEQ ID No. 3.
50. A heterologous host cell comprising a nucleic acid according to any one of items 46-49, wherein the nucleic acid is recombinant.
51. The heterologous host cell according to item 50, wherein the cell is a yeast cell, a plant cell, a mammalian cell, an insect cell, a fungal cell, a bacterial cell, an algal cell, or a cyanobacterial cell.
52. The heterologous host cell according to any one of items 50-51, wherein the cell is a yeast cell selected from the group consisting of Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.
53. The heterologous host cell according to any one of items 50-52, wherein the host cell is a Saccharomyces.
54. The heterologous host cell according to any one of items 50-53, wherein the host cell is a yeast cell is a Saccharomyces cerevisiae cell.
55. A method for the preparation of a (S)-norcoclaurine and/or (S)-norlaudanosoline compound, the method comprising the steps of:
   providing a recombinant host cell according to any of items 50-54 capable of catalysing of the condensation of 4-HPAA and dopamine to (S)-norcoclaurine and/or 3,4-DhPAA and dopamine to (S)-norlaudanosoline,
   culturing the cell under conditions promoting said catalysation, and optionally
   isolating the (S)-norcoclaurine and/or (S)-norlaudanosoline.
56. A method for the preparation of a (S)-norcoclaurine and/or (S)-norlaudanosoline compound, comprising contacting compound 4-HPAA and dopamine and/or 3,4-DhPAA and dopamine with a recombinant norcoclaurine synthase according to any of items 42-49 capable of catalysation of the condensation of 4-HPAA and dopamine to (S)-norcoclaurine and/or 3,4-DhPAA and dopamine to (S)-norlaudanosoline.
57. The method according to item 56, further comprising cultivating a recombinant host cell of any one of items 50-54 in a culture medium in presence of 4-HPAA and dopamine and/or 3,4-DhPAA and dopamine, under conditions in which the one or more recombinant genes encoding the recombinant norcoclaurine synthase according to any of items 42-49 is/are expressed in presence of 4-HPAA and dopamine and/or 3,4-DhPAA and dopamine.
58. The method according to claim 56-57, which is performed in vitro.
59. An opioid selected from the group consisting of Thebaine, Oripavine, Neopinone, Codeinone, Hydrocodone, Morphine, Oxycodone, Codeine, Noscapine, Berberine, Sanguinarine, Tubocurarine and Papaverine obtainable from a method according to any of items 55-58 or 24-27.

1-15. (canceled)

16. A method for preparing a composition of matter comprising a benzylisoquinoline alkaloid (BIA) from a (S)-norcoclaurine and/or (S)-norlaudanosoline substrate comprising the steps of:
a) providing a recombinant host cell comprising a variant norcoclaurine synthase capable of catalyzing the condensation of 4-hydroxy-phenylacetaldehyde (4-HPAA) and dopamine into (S)-norcoclaurine and/or 3,4-dihydroxy-acetaldehyde (3,4-DhPAA) and dopamine into (S)-norlaudanosoline, wherein the variant norcoclaurine synthase comprises:
i) one or more site-specific substitutions corresponding to positions 73, 77, 99, 114, 142, 152, 174, and 178 of SEQ ID NO: 1; or
ii) one or more site-specific substitutions corresponding to positions 73, 77, 99, 114, 142, 152, 174, and/or 178 of SEQ ID NO: 1, and an N-terminal truncation; and
b) culturing the recombinant host cell under conditions promoting said catalyzation; and optionally
c) isolating the BIA.

17. The method according to claim 16, wherein the variant norcoclaurine synthase comprises a heterologous amino acid sequence which is at least 50% identical to a norcoclaurine synthase comprised in the sequence of SEQ ID NO: 1, or an N-terminal truncation thereof.

18. The method according to claim 16, wherein the one or more site-specific substitutions is one or more of A73P, A77S, A77E, A77T, Q99K, Q99R, K114E, V142I, K152R, V174E, V174G, V174Q, I178A, I178S, I178D, I178N, I178Q, and I178T of SEQ ID NO: 1.

19. The method according to claim 16, wherein the one or more site-specific substitutions are at positions i) 141, 142 and 152; ii), 75 and 178; or iii) 75, 152, 178 of SEQ ID NO: 1.

20. The method according to claim 19, wherein the one or more site-specific substitutions are i) V141I, V142I and K152R; ii) I75L, I178D or I75K, I178D; or iii) I75K, K152R and I178D of SEQ ID NO: 1.

21. The method according to claim 16, wherein the variant norcoclaurine synthase comprises an endoplasmic reticulum (ER) retention signal at the C-terminus of the variant norcoclaurine synthase.

22. The method according to claim 21, wherein the ER retention signal comprises the amino acid sequence HDEL (SEQ ID NO: 22) at the C-terminus of the variant norcoclaurine synthase.

23. The method according to claim 16, wherein the variant norcoclaurine synthase comprises the N-terminal truncation.

24. The method according to claim 23, wherein the N-terminal truncation comprises a substitution or a deletion of one or more amino acids between residues 1-19 of SEQ ID NO: 1; wherein signal peptide function is disrupted.

25. The method according to claim 23, wherein the N-terminal truncation comprises a deletion of residues 1-19 of SEQ ID NO: 1.

26. The method according to claim 16, wherein the variant norcoclaurine synthase has an increased catalyzation of the condensation of 4-hydroxy-phenylacetaldehyde (4-HPAA) and dopamine into (S)-norcoclaurine and/or 3,4-dihydroxy-acetaldehyde (3,4-DhPAA) and dopamine into (S)-norlaudanosoline when compared to a wild-type synthase of SEQ ID NO: 1.

27. The method according to claim 16, wherein the recombinant host cell comprises a nucleic acid sequence encoding a norcoclaurine synthase, wherein the nucleic acid sequence is at least 70% identical to a nucleic acid sequence of SEQ ID NO: 2 encoding a norcoclaurine synthase.

28. The method according to claim 27, wherein the nucleic acid sequence is codon optimized for S. cerevisiae.

29. The method according to claim 27, wherein the nucleic acid sequence is at least 60% identical to the nucleic acid sequence of SEQ ID NO: 3.

30. The method according to claim 29, wherein the nucleic acid sequence is at least 80% identical to the nucleic acid sequence of SEQ ID NO: 3.

31. The method according to claim 16, wherein the recombinant host cell is a yeast cell, a plant cell, a mammalian cell, an insect cell, a fungal cell, a bacterial cell, an algal cell, or a cyanobacterial cell.

32. The method according to claim 31, wherein the recombinant host cell is a yeast cell selected from the group consisting of Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.

33. The method according to claim 32, wherein the recombinant host cell is a Saccharomyces.

34. The method according to claim 33, wherein the recombinant host cell is a yeast cell is a Saccharomyces cerevisiae cell.

35. The method according to claim 16, wherein the BIA is selected from the group consisting of Thebaine, Oripavine, Neopinone, Codeinone, Hydrocodone, Morphine, Oxycodone, Codeine, Noscapine, Berberine, Sanguinarine, Tubocurarine, and Papaverine.

36. A benzylisoquinoline alkaloid (BIA) produced by the method of claim 16.

37. The BIA according to claim 36, wherein the BIA is thebaine, oripavine, neopinone, codeinone, hydrocodone, morphine, oxycodone, codeine, noscapine, berberine, sanguinarine, tubocurarine, or papaverine.
